SEQUENCE LISTING (24 sequences)

SEQ ID NO: 1 - DNA, 12 bp, Artificial Sequence
  synthetic oligo
ggcgcgagtttc

SEQ ID NO: 2 - DNA, 78 bp, Artificial Sequence
  synthetic oligo
ctcctcgcggcgcgagtttcaggcagcgctgcgtcctgctgcgcacgtgggaagccctggccccggccacccccgcga

SEQ ID NO: 3 - DNA, 11 bp, Artificial Sequence
  synthetic oligo
ggcgcgagttt

SEQ ID NO: 4 - DNA, 634 bp, Artificial Sequence
  plasmid sequence
aaagacttttcaccaaaaaatgtcatttatgatgactcatcccagtatttgatcatggaaagaattctaagtcaaggccctgtgtattccagttttaaaggaggctggaaatgcaaggatcatactgagatgctgcaagaaaatcagggatgtattaggaaagtaacagtctytcatcaagaarccctggctcaacatatgaatatcagtactggggagaggccctatggatgccatgaatgtggaaaaacttttggtcgacgcttttccctggkgttacaccagaggactcatactggagagaaaccatatgcatgtaaggaatgagtgatttgggaatgccttcatttacaagcaatacttaaattttaaactcttgtaggaraaaaagcactgmataaatgaatgtagagtgacyttttgcaatatttcaacctatatcaagaattacctgtgggraaactaccttgtaataagmgtagcaaaatctccttaraatytgaaaagtcatactggatggaatctgtaggaaacggttytattttmarggaagggggattccyttttgttttttaagtgaattcaraaaatgttataaataaatcttttggtttttataaaaaaaaaaaaaaaa

SEQ ID NO: 5 - DNA, 984 bp, Artificial Sequence
  plasmid sequence
tggaaatgcaaggatcatactgagatgctgcaagaaaatcagggatgtattaggaaagtaacagtctctcatcaagaagccctggctcaacatatgaatatcagtactgtggagaggccctatggatgccatgaatgtggaaaaacttttggtcgacgcttttccctggtgttacaccagaggactcatactggagagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcagagaattcacataggaaagaaacaatatatatgtaggaaatgtggtaaagcatttagcagtggctcaaaactcattcgccaggaaagaatcatatccaatagattggagaaagccagagattagccctcattccgcatctgtcaaccaggacagaatgcatggcaagggatgagctttacaaagatgatgcctttggagatcagaaaattcatatttaagcaaagtgatacaaaccagtgatttgggaatgccttcatttacaatgcaatacttaaattttaatactcttgtaggagaaaaagcactgtataaatgaatgtagagtgactttctgcaatatttcaaacctatatcagagaattacactgtgggaaaactaccattgtaataagtgtagcaaaatctccttagatatctgaaaagtcatactggatggaatctgtaggaaacggttctattttgagggaagggggattcctttttgttttttaagtgaattcagaaaatgttataaataaatcttttggtttattataaaaaaaaaaaaaaaaa

SEQ ID NO: 6 - DNA, 1047 bp, Artificial Sequence
  plasmid sequence
gagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcagagaattcacataggaaagaaacaatatatatgtaggaaatgtggtaaagcatttagcagtggctcaraactcattcgccaccagattacacatactggagagaaaccttatgaatgcattgaatgkgggaaggcatttcgccgtttctcacaccttactcgacatcagagcatycatacaaccaaaaccccgtatgaatgtaatgaatgkaggaaagctttccgtgtcactcatttcttattaaacatcaaaaaattcatgctggagaaaagctctatgaatgaaaaaaaaaaaaaaaaaatggaaatgcaaggatcatactgagatgctgcaagaaaatcagggatgtattaggaaagtaacagtctctcatcaagaagccctggctcaacatatgaatatcagtactgtggagaggccctatggatgccatgaatgtggaaaaacttttggtcgacgcttttccctggtgttacaccagaggactcatactggagagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcagagaattcacataggaaagaaacaatatatatgtaggaaatgtggtaaagcatttagcagtggctcaaaactcattcg

SEQ ID NO: 7 - DNA, 677 bp, Artificial Sequence
  plasmid sequence
cacgtatgtgagttctgcagccacgccttcacccaaaaggccaatctcaacatgcacctgcgcacacacacgggtgagaagcccttccagtgccacctctgtggcaagaccttccgaacccaagccagcctggacaagcacaaccgcacccacaccggggaaaggcccttcagttgcgagttctgtgaacagcgcttcactgagaaggggcccctcctgaggcacgtggccagccgccatcaggagggccggccccacttctgccagatatgcggcaagaccttcaaagccgtggagcaactgcgtgtgcacgtcaracggcacaagggggtgaggaagtttgagtgcaccgagtgtggctacaagtttacccgacaggcccacctgcggaggcacatggagatatggtggtggtggcgctgcarccgcctgcararctggaggtgggctcggcggaggtcattgtggagtccctggcccagggcggcctggcctcccagctccccggccararactgtgtgcaaaggaragcttyccggcccaggtgtcctggagccctccctcatcatcacagctgctgtccccgaggactgtgacacatagcccattytggccccaragccccttggcccccccctcaataaaccgtgtggctttggaaaaaaaaaaaaaaaaa

SEQ ID NO: 8 - DNA, 526 bp, Artificial Sequence
  plasmid sequence
ctctgtggcaagaccttccgaacccaagccagcctggacaagcacaaccgcacccacaccggggaaaggcccttcagttgcgagttctgtgaacagcgcttcactgagaaggggcccctcctgaggcacgtggccagccgccatcaggagggccggccccacttctgccagatatgcggcaagaccttcaaagccgtggagcaactgcgtgtgcacgtcagacggcacaagggggtgaggaagtttgagtgcaccgagtgtggctacaagtttacccgacaggcccacctgcggaggcacatggagatccacgaccgggtagagaactacaacccgcggcagcgcaagctccgcaacctgatcatcgaggacraraagatggtggtggtggcgctgcagccgcctgcaragctggaggtgggctcggcggaggtcattgtggagtccctgcccagggcggcctgcctccagctccccggccagaractgtgtgcaaaggaaaacttcaaaaaaaaaaaaaaaaaaa

SEQ ID NO: 9 - DNA, 522 bp, Artificial Sequence
  plasmid sequence
gggcaccgggcctcgagccggaatggcctgcagatgcacatcaaggccaagcacaggaatgagaggccacacgtatgtgagttctgcagccacgccttcacccaaaaggccaatctcaacatgcacctgcgcacacacacgggtgagaagcccttccagtgccacctctgtggcaagaccttccgaacccaagccagcctggacaagcacaaccgcacccacaccggggaaaggcccttcagttgcgagttctgtgaacagcgcttcactgagaaggggcccctcctgaggcacgtggccagccgccatcaggagggccggccccacttctgccagatatgcggcaagaccttcaaagccgtggagcaactgsgtgtgcacgtcaaaacggcacaagggggtgaggaaagttgagtgcaccgagtgtggctacaaatttacccgacaggcccacttggggaggcacatggaaatcccgaccgggtaaaaaactacaaccccgaaaaaaaaaaaaaaaaaaaa

SEQ ID NO: 10 - DNA, 534 bp, Artificial Sequence
  plasmid sequence
atcaaacttcatggagcccccaagccccatgcatgccccacctgtgccaagtgcttctgtctcggacagagctgcagctgcatgaagctttcaagcaccgtggtgagaagctgtttgtgtgtgaggagtgtgggcaccgggcctcgagccggaatggcctgcagatgcacatcaaggccaagcacaggaatgagaggccacacgtatgtgagttctgcagccacgccttcacccaaaaggccaatctcaacatgcacctgcgcacacacacgggtgagaagcccttccagtgccacctytgtggcaagaccttycgaacccaagccagcctggacaagcacaaccgcacccacaccggggaaaggcccttcagttgcgagttctgtgaacagcgcttmactgagaaggggcccctyctgaggcacgtggccagccgccatyaggagggccggccccacttytgccagatatgcggcaagaccttyaaagccgtggagcaactgggtgtgcacgtaaaaaaaaaaaaaaaaaaaa

SEQ ID NO: 11 - DNA, 483 bp, Artificial Sequence
  plasmid sequence
gaagcccggaattgcatgaaccgctcggaacaggtcttcacgtgctctgtgtgccaggagacattccgccgaaggatggagctgcgggtgcacatggtgtctcacacaggggagatgccctacaagtgttcctcctgctcccagcagttcatgcagaagaaggacttgcagagccacatgatcaaacttcatggagcccccaagccccatgcatgccccacctgtgccaagtgcttcctgtctcggacagagctgcagctgcatgaagctttcaagcaccgtggtgagaagctgtttgtgtgtgaggagtgtgggcaccgggcctcgagccggaatggcctgcagatgcacatcaaggccaagcacaggaatgagaggccacacgtatgtgagttctgcagccacgccttcacccaaaaggccaatctcaacatgcacctgcgcacacacacgggtgagaatcccttccagtgccacctttgg

SEQ ID NO: 12 - DNA, 13 bp, Artificial Sequence
  plasmid sequence
gatccgcaccttg

SEQ ID NO: 13 - DNA, 16 bp, Artificial Sequence
  plasmid sequence
gatccctttacccagg

SEQ ID NO: 14 - DNA, 532 bp, Artificial Sequence
  plasmid sequence
cagacccctcaagccttaacatttaatgatgggatgagtgaagtgaaagatgaacagacaccaggctggacaacagccgccagtgacatgaagtttgagtatttgctttatggtcaccatcgggagcagattgcctgccaggcgtgtgggaagacgttttctgatgaaggcagattgaggaagcatgagaaactccacacggcggacaggccatttgtttgtgaaatgtgcacaaaaggtttcaccacacaggcccacctgaaagaacacctaaaaatccacacaggatataagccctatagctgtgaggtgtgtggaaaatcatttatccgtgccccagacttaaagaagcatgagagagttcacagtaatgaaagaccgtttgcgtgccacatgtgtgacaaagccttcaaacacaagtctcacctyaaggatcatgaaagaagacacagaggggaaaagccttttgtgtgtggytcctgcaccaaggcatttgccattttagtgtgaaaaaaaaaaaaaaaaaaaaaaa

SEQ ID NO: 15 - DNA, 877 bp, Artificial Sequence
  plasmid sequence
cagacccctcaagccttaacatttaatgatgggatgagtgaagtgaaagatgaacagacaccaggctggacaacagccgccagtgacatgaagtttgagtatttgctttatggtcaccatcgggagcagattgcctgccaggcgtgtgggaagacgttttctgatgaaggcagattgaggaagcatgagaaactccacacggcggacaggccatttgtttgtgaaatgtgcacaaaaggtttcaccacacaggcccacctgaaagaacacctaaaaatccacacaggatataagccctatagctgkgaggkgtgtggaaaatcatttatccgtgccccagacttaaagaagcatgagagagttcacagtaatgaaagaccgtttgcgtgccacatgtgtgacaaagccttcaaacacaagtctyacctyaaggrtcatgaaagaagacctacccaaaagctgtagtcacacatcctaaaggccaarcaaacccccgggatggtggggggtcttggarccaagctyttaggttcctcttatttggggcagmaccagtccataccagctgcgatttgtgagtggacctgtggtaagaagaatagaaaaggctctcararataaggttttttacatgtgtaacaatcccaagatttcctagattaaaatyttaattgattttgaaattggatttttatttagaatcaaaattaggrcaagaacagataacttcttcaratacatttgtgtaactttacagaatgtcatcaagctttggggctctgtggggcacatgatttatccataaaggagatgcagtatgcttacttaaattaataaatttaaaatcttttaagtgtgaaaaaaaaaaaaaaaaaaa

SEQ ID NO: 16 - DNA, 603 bp, Artificial Sequence
  plasmid sequence
agtgacatgaagtttgagtatttgctttatggtcaccatcgggagcagattgcctgccaggcgtgtgggaagacgttttctgatgaaggcagattgaggaagcatgagaaactccacacggcggacaggccatttgtttgtgaaatgtgcacaaaaggtttcaccacacaggcccacctgaaagaacacctaaaaatccacacaggatataagccctatagctgtgaggtgtgtggaaaatcatttatccgtgccccagacttaaagaagcatgagagagttcacagtaatgaaagaccgtttgcgtgccacatgtgtgacaaagccttcaaacacaagtctcacctcaaggatcatgaaagaagacacagaggggaaaagccttttgtgtgtggctcctgcaccaaggcatttgccaaggcatctgatctgaaaaggcacgagaacaatatgcacagtgaaaggaagcaggttacccccagtgccatccagagcgagacagaacagttgcaggcggcagcgatggctgcggaagcagaacagcagctggagacgatagyctgtaactagaggcggtgggacagggacactttgcctggaaag

SEQ ID NO: 17 - DNA, 8 bp, Artificial Sequence
  oligo
gsgcgcgr

SEQ ID NO: 18 - DNA, 71 bp, Artificial Sequence
  oligo
gaattcggcgcgagtttagggcgcgagtttagggcgcgagtttagggcgcgagtttagggcgcgagtttag

SEQ ID NO: 19 - protein, 457 aa, human
MSQGSVTFRDVAIDFSQEEWKWLQPAQRDLYRCVMLENYGHLVSLGLSISKPDVVSLLEQGKEPWLGKREVKRDLFSVSESSGEIKDFSPKNVIYDDSSQYLIMERILSQGPVYSSFKGGWKCKDHTEMLQENQGCIRKVTVSHQEALAQHMNISTVERPYGCHECGKTFGRRFSLVLHQRTHTGEKPYACKECGKTFSQISNLVKHQMIHTGKKPHECKDCNKTFSYLSFLIEHQRTHTGEKPYECTECGKAFSRASNLTRHQRIHIGKKQYICRKCGKAFSSGSELIRHQITHTGEKPYECIECGKAFRRFSHLTRHQSIHTTKTPYECNECRKALRCHSFLIKHQRIHAGEKLYECDECGKVFTWHASLIQHTKSHTGEKPYACAECDKAFSRSFSLILHQRTHTGEKPYVCKVCNKSFSWSSNLAKHQRTHTLDNPYEYENSFNYHSFLTEHQ

SEQ ID NO: 20 - DNA, 2407 bp, human
ctaaaggtcggcgaggcttctgaagacgcaattcctgcgacgcccgcggaggggccctggggggcggcgcgagcgtctggcctgtgttggctgtaggcaacgaaaggagccctcccggtctgcgccggatggccccgggcggtgactcggtccggagccctggaacgctacgcccacctggcggaaagcaccacggaaacgcatccttctgtggccactgttaggtctgccattttacacttttctgatctcctccttcccttctgtgagctatgtctcaggggtcagtgacattcagagatgtggccatagacttctcccaggaggagtggaaatggcttcagcctgctcaaagagatttgtacagatgtgtaatgttggagaactatggccatctggtctcactgggtctttccatttctaagccagatgtggtttccttattggagcaagggaaagaaccctggctggggaaaagggaagtgaaaagagatctgttttcagtttcagagtcaagtggtgagatcaaagacttttcaccaaaaaatgtcatttatgatgactcatcccagtatttgatcatggaaagaattctaagtcaaggccctgtgtattccagttttaaaggaggctggaaatgcaaggatcatactgagatgctgcaagaaaatcagggatgtattaggaaagtaacagtctctcatcaagaagccctggctcaacatatgaatatcagtactgtggagaggccctatggatgccatgaatgtggaaaaacttttggtcgacgcttttccctggtgttacaccagaggactcatactggagagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcaaagaattcacataggaaagaaacaatatatatgtaggaaatgtggtaaagcatttagcagtggctcagaactcattcgccaccagattacacatactggagagaaaccttatgaatgcattgaatgtgggaaggcatttcgccgtttctcacaccttactcgacatcagagcatccatacaaccaaaaccccgtatgaatgtaatgaatgtaggaaagctttgcgttgtcactcattccttattaaacatcagagaattcatgctggagaaaagctctatgaatgtgatgaatgtggtaaagttttcacttggcatgcatcccttattcaacatacgaagagtcacactggagagaaaccctatgcgtgtgctgaatgtgataaagccttcagccggagcttttccctcattctacatcagagaactcatactggagagaaaccctatgtatgtaaggtatgcaacaaatccttcagctggagctcaaaccttgctaaacatcagaggacacacactcttgacaacccctatgaatatgaaaattcatttaattaccactcattccttactgaacaccagtgaatttacactgcaaagaaaaactatgaatgtatggaattttttaaaaagaagtataatgccttacttcagagaactcttggaaagaagccttatgtgaaagtgatgactgtgaagtaatatggcccacactttattcaccaccctggagaaaaaaaaacccaggaatatgtggaaaagccattaataaccactcttttatttttttgcaataacaaggtgaaatcaatattgttgagaagattcttccatctggtaatgttgagaagacttcatttggtaggagtcccttactttacgtgtgtaaattcctaccaggaaagaatacatatccaatagattggagaaagccagagattagccccgcattccgcatctgtcaaccaggacagaaagcatggacaagggatgagctttacaaagatgatgcactttggagatcagaaaattcatatttaagcaaagtgatacaaacacagtgatttgggaatgccttcatttacaatgcaatacttacattttaatactcttgtaggagaaaaagcaactgtataaatgaatgtagagtgactttctgcaatatttgcaacctatatcagagaattacactgtgggaaaactaccattgtaataagtgtagcaaaatctccttagatatctgaaaagtcatactggatggaatctgtaggaaacggttctattttgagggaagggggattcctttttgttttttaagtgaattcagaaaatgttataaactttag

SEQ ID NO: 21 - protein, 688 aa, human
MDGSFVQHSVRVLQELNKQREKGQYCDATLDVGGLVFKAHWSVLACCSHFFQSLYGDGSGGSVVLPAGFAEIFGLLLDFFYTGHLALTSGNRDQVLLAARELRVPEAVELCQSFKPKTSVGQAAGGQSGLGPPASQNVNSHVKEPAGLEEEEVSRTLGLVPRDQEPRGSHSPQRPQLHSPAQSEGPSSLCGKLKQALKPCPLEDKKPEDCKVPPRPLEAEGAQLQGGSNEWEVVVQVEDDGDGDYMSEPEAVLTRRKSNVIRKPCAAEPALSAGSLAAEPAENRKGTAVPVECPTCHKKFLSKYYLKVHNRKHTGEKPFECPKCGKCYFRKENLLEHEARNCMNRSEQVFTCSVCQETFRRRMELRVHMVSHTGEMPYKCSSCSQQFMQKKDLQSHMIKLHGAPKPHACPTCAKCFLSRTELQLHEAFKHRGEKLFVCEECGHRASSRNGLQMHIKAKHRNERPHVCEFCSHAFTQKANLNMHLRTHTGEKPFQCHLCGKTFRTQASLDKHNRTHTGERPFSCEFCEQRFTEKGPLLRHVASRHQEGRPHFCQICGKTFKAVEQLRVHVRRHKGVRKFECTECGYKFTRQAHLRRHMEIHDRVENYNPRQRKLRNLIIEDEKMVVVALQPPAELEVGSAEVIVESLAQGGLASQLPGQRLCAEESFTGPGVLEPSLIITAAVPEDCDT

SEQ ID NO: 22 - DNA, 2289 bp, human
tacgcatagccgggcactaggttcgtgggctgtggaggcgacggagcagggggccagtggggccagctcagggaggacctgcctgggagctttctcttgcataccctcgcttaggctggccggggtgtcacttctgcctccctgccctccagaccatggacggctccttcgtccagcacagtgtgagggttctgcaggagctcaacaagcagcgggagaagggccagtactgcgacgccactctggacgtggggggcctggtgtttaaggcacactggagtgtccttgcctgctgcagtcactttttccagagcctctacggggatggctcagggggcagtgtcgtcctccctgctggcttcgctgagatctttggcctcttgttggactttttctacactggtcacctcgctctcacctcagggaaccgggatcaggtgctcctggcagccagggagttgcgagtgccagaggccgtagagctgtgccagagcttcaagcccaaaacttcagtgggacaggcagcaggtggccagagtgggctggggccccctgcctcccagaatgtgaacagccacgtcaaggagccggcaggcttggaagaagaggaagtttcgaggactctgggtctagtccccagggatcaggagcccagaggcagtcatagtcctcagaggccccagctccattccccagctcagagtgagggcccctcctccctctgtgggaaactgaagcaggccttgaagccttgtccccttgaggacaagaaacccgaggactgcaaagtgcccccaaggcccttagaggctgaaggtgcccagctgcagggcggcagtaatgagtgggaagtggtggttcaagtggaggatgatggggatggcgattacatgtctgagcctgaggctgtgctgaccaggaggaagtcaaatgtaatccgaaagccctgtgcagctgagccagccctgagcgcgggctccctagcagctgagcctgctgagaacagaaaaggtacagcggtgccggtcgaatgccccacatgtcataaaaagttcctcagcaaatattatctaaaagtccacaacaggaaacatactggggagaaaccctttgagtgtcccaaatgtgggaagtgttactttcggaaggagaacctcctggagcatgaagcccggaattgcatgaaccgctcggaacaggtcttcacgtgctctgtgtgccaggagacattccgccgaaggatggagctgcgggtgcacatggtgtctcacacaggggagatgccctacaagtgttcctcctgctcccagcagttcatgcagaagaaggacttgcagagccacatgatcaaacttcatggagcccccaagccccatgcatgccccacctgtgccaagtgcttcctgtctcggacagagctgcagctgcatgaagctttcaagcaccgtggtgagaagctgtttgtgtgtgaggagtgtgggcaccgggcctcgagccggaatggcctgcagatgcacatcaaggccaagcacaggaatgagaggccacacgtatgtgagttctgcagccacgccttcacccaaaaggccaatctcaacatgcacctgcgcacacacacgggtgagaagcccttccagtgccacctctgtggcaagaccttccgaacccaagccagcctggacaagcacaaccgcacccacaccggggaaaggcccttcagttgcgagttctgtgaacagcgcttcactgagaaggggcccctcctgaggcacgtggccagccgccatcaggagggccggccccacttctgccagatatgcggcaagaccttcaaagccgtggagcaactgcgtgtgcacgtcagacggcacaagggggtgaggaagtttgagtgcaccgagtgtggctacaagtttacccgacaggcccacctgcggaggcacatggagatccacgaccgggtagagaactacaacccgcggcagcgcaagctccgcaacctgatcatcgaggacgagaagatggtggtggtggcgctgcagccgcctgcagagctggaggtgggctcggcggaggtcattgtggagtccctggcccagggcggcctggcctcccagctccccggccagagactgtgtgcagaggagagcttcaccggcccaggtgtcctggagccctccctcatcatcacagctgctgtccccgaggactgtgacacatagcccattctggccaccagagcccacttggccccacccctcaataaaccgtgtggctttggactctcgt

SEQ ID NO: 23 - protein, 449 aa, human
MEFFVSMSETIKYNDDDHKTLFLKTLNEQRLEGEFCDIAIVVEDVKFRAHRCVLAACSTYFKKLFKKLEVDSSSVIEIDFLRSDIFEEVLNYMYTAKISVKKEDVNLMMSSGQILGIRFLDKLCSQKRDVSSPDENNGQSKSKYCLKINRPIGDAADTQGCDVEEIGDQDDSPSDDTVEGTPPSQEDGKSPTTTLRVQEAILKELGSEEVRKVNCYGQEVESMETPESKDLGSQTPQALTFNDGMSEVKDEQTPGWTTAASDMKFEYLLYGHHREQIACQACGKTFSDEGRLRKHEKLHTADRPFVCEMCTKGFTTQAHLKEHLKIHTGYKPYSCEVCGKSFIRAPDLKKHERVHSNERPFACHMCDKAFKHKSHLKDHERRHRGEKPFVCGSCTKAFAKASDLKRHENNMHSERKQVTPSAIQSETEQLQAAAMAAEAEQQLETIACS

SEQ ID NO: 24 - DNA, 1861 bp, human
gcgttcaaggcattaagataatagcctgagttgttcatggagtttttcgtcagtatgtctgaaaccattaaatataatgacgatgatcataaaactctgtttctgaaaacactaaatgaacaacgcctggaaggagaattttgtgatattgctattgtggttgaggatgtgaaattcagagcacacagatgtgttcttgctgcctgcagcacctactttaaaaagcttttcaagaagcttgaggttgatagttcttcggtcatagaaatagattttcttcgttctgatatatttgaagaggtcctgaactacatgtacacagcaaagatttccgtgaaaaaagaagatgttaacttaatgatgtcatcgggtcagattcttggtatccgatttttggataaactgtgttctcagaagcgtgatgtgtccagtcccgatgaaaacaatggtcagtccaaaagtaagtattgccttaaaataaatcgccccattggagatgctgctgacacccagggatgtgatgtagaggaaatcggggatcaggatgacagtccttctgatgacacagtagaaggcacacccccgagtcaggaggacggcaagtcgcccaccacaacgctcagggttcaggaagcgatcctgaaagagctggggagtgaggaagttcggaaggtcaattgctacggccaggaagtagaatccatggagaccccagaatcaaaagacttggggtcccagacccctcaagccttaacatttaatgatgggatgagtgaagtgaaagatgaacagacaccaggctggacaacagccgccagtgacatgaagtttgagtatttgctttatggtcaccatcgggagcagattgcctgccaggcgtgtgggaagacgttttctgatgaaggcagattgaggaagcatgagaaactccacacggcggacaggccatttgtttgtgaaatgtgcacaaaaggtttcaccacacaggcccacctgaaagaacacctaaaaatccacacaggatataagccctatagctgtgaggtgtgtggaaaatcatttatccgtgccccagacttaaagaagcatgagagagttcacagtaatgaaagaccgtttgcgtgccacatgtgtgacaaagccttcaaacacaagtctcacctcaaggatcatgaaagaagacacagaggggaaaagccttttgtgtgtggctcctgcaccaaggcatttgccaaggcatctgatctgaaaaggcacgagaacaatatgcacagtgaaaggaagcaggttacccccagtgccatccagagcgagacagaacagttgcaggcggcagcgatggctgcggaagcagaacagcagctggagacgatagcctgtagctagaggcggtgggacagggacactttgcctggaaagtggagactgagatgacgtggatcataatgagtgaatgccagttacaatatttttgtggaaacgtatggaacattgtactcactggacttaaggcagtgcttggttagctatttttaagacttttcaaggaaatggtgttcctcagttctgaccaaacgtttcactgtcttgtctggtgtctagtattaatgttgccagtaagcacctctctcccttttttttttttttattattttaatttgagaactcctgtgtccagtttagaagtgagagacttccatttttagttcctttacactcaccaccctagcaagtgccctgcacagagtaataagtaaattgatttcctaatcacaattctatgtgacttatggtcaaaacagcagtcgagaaaaatcactttttctttcatcatacacacctctaaaaagaactggaattc

(19) United States
(12) Patent Application Publication
(10) Pub. No.: US 2005/0176629 A1
(43) Pub. Date: Aug. 11, 2005

(54) Telomerase expression repressor proteins and methods of using the same

(76) Inventors: William H. Andrews, Reno, NV (US); Christopher A. Foster, Reno, NV (US); Stephane Fraser, Sparks, NV (US); Hamid Mohammadpour, Reno, NV (US); Laura Briggs, Reno, NV (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/477,646

(22) PCT Filed: Mar. 12, 2002
(86) PCT No.: PCT/US02/07918

Related U.S. Application Data
(60) Provisional application No. 60/275,691, filed on Mar. 13, 2001.
(60) Provisional application No. 60/275,681, filed on Mar. 13, 2001.
(60) Provisional application No. 60/275,689, filed on Mar. 13, 2001.

(51) Int. Cl. A61K 38/17

(52) U.S. Cl. 514/12

(57) ABSTRACT

Telomerase repressor proteins and nucleic acid compositions encoding the same are provided. The subject repressor proteins bind to a repressor site in the TERT minimal promoter, e.g., a Site C site, and thereby inhibit TERT expression. Also provided are methods of modulating, e.g., inhibiting or enhancing, TERT expression. The subject invention finds use in a variety of different applications, including therapeutic and therapeutic agent screening applications.

### CROSS-REFERENCE TO RELATED APPLICATIONS

Pursuant to 35 U.S.C. § 119 (e), this application claims priority to the filing date of the U.S. Provisional Patent Application Ser. Nos.: (a) 60/275,691filed Mar. 13, 2001; (b) 60/275,681 filed Mar. 13, 2001; and (c) 60/275,689 filed Mar. 13, 2001; the disclosures of which are herein incorporated by reference.

### INTRODUCTION

1. Field of the Invention

The field of this invention is the telomerase reverse transcriptase gene, specifically the regulation of the expression thereof.

2. Background of the Invention

Telomeres, which define the ends of chromosomes, consist of short, tandemly repeated DNA sequences loosely conserved in eukaryotes. Human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. Human telomerase is made up of two components: (1) an essential structural RNA (TER) (where the human component is referred to in the art as hTER); and (2) a catalytic protein (telomerase reverse transcriptase or TERT) (where the human component is referred to in the art as hTERT). Telomerase works by recognizing the 3′ end of DNA, e.g., telomeres, and adding multiple telomeric repeats to its 3′ end with the catalytic protein component, e.g., hTERT, which has polymerase activity, and hTER which serves as the template for nucleotide incorporation. Of these two components of the telomerase enzyme, both the catalytic protein component and the RNA template component are activity limiting components.

Because of its role in cellular senescence and immortalization, there is much interest in the development of protocols and compositions for regulating expression of telomerase.

### RELEVANT LITERATURE

U.S. patents of interest include: U.S. Pat. Nos. 6,093,809; 6,054,575; 6,013,468; 6,007,989; 5,958,680; 5,876,979; 5,858,777; 5,837,857; 5,583,016; 4,816,397; 4,816,567; 5,693,780; 5,681,722; 5,658,570; 5,750,105; 5,756,096; 5,464,764; and 5,627,052. Also of interest are WO 99/33998 and WO 99/35243. Articles of interest include: Takakura et al., Cancer Res. (1999) 59:551-7; Cong et al., Hum. Mol. Genet. (1999) 8:137-142; Wu et al., Nat. Genet. (1999) 21:220-224; and Horikawa et al., Abstract # 1429, Scientific Proceedings, 91^{st} Annual Meeting of American Association for Cancer Research, San Francisco, Calif. Apr. 1-5, 2000. See also GENBANK accession nos. AF1 14847 and 128893. Articles discussing ZNF140 transcription factors include: FEBS Left. Aug. 7, 1995; 369 (2-3): 153-7, Genomics 27 (2), 259-264 (1995). Articles discussing HKR3 transcription factors include: Maris J. M. et al., Genomics 15; 35 (2): 289-298, 1996, White, P. S. et al., Eur J. Cancer 33 (12):1957-1961, 1997. Articles discussing ZFP161 and ZF5 transcription factors include: Sugiura K. et al., Biochim. Biophys. Acta 1352 (1): 23-26, 1997, Sobe-Klocke, I. et al., Genomics 43(2):156-164, 1997. All of the patents and publications cited are incorporated herein by reference in their entirety.

### SUMMARY OF THE INVENTION

Telomerase repressor proteins and nucleic acid compositions encoding the same are provided. The subject repressor proteins bind to a repressor site in the TERT minimal promoter, e.g., a Site C site, and thereby inhibit TERT expression. Also provided are methods of modulating, e.g., inhibiting or enhancing, TERT expression. The subject invention finds use in a variety of different applications, including therapeutic and therapeutic agent screening applications.

### BRIEF DESCRIPTION OF THE DRAWINGS

FIG. 1 provides the amino acid and nucleotide sequence of human ZNF140.

FIG. 2 provides the amino acid and nucleotide sequence of human HKR3.

FIG. 3 provides the amino acid and nucleotide sequence of human ZFP161.

### DESCRIPTION OF THE SPECIFIC EMBODIMENTS

Telomerase repressor proteins and nucleic acid compositions encoding the same are provided. The subject repressor proteins bind to a repressor site in the TERT minimal promoter, e.g., a Site C site, and thereby inhibit TERT expression. Also provided are methods of modulating, e.g., inhibiting or enhancing, TERT expression. The subject invention finds use in a variety of different applications, including therapeutic and therapeutic agent screening applications. In further describing the subject invention, the subject polypeptide and nucleic acid compositions are described first, followed by a discussion of various utilities of the subject applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the elements that are described in the publications which might be used in connection with the presently described invention.

Telomerase Repressor Polypeptide Compositions

As summarized above, the subject invention provides telomerase repressor polypeptides, i.e., polypeptides that repress telomerase expression. The term polypeptide composition as used herein refers to both full length proteins as well as portions or fragments thereof. Also included in this term are variations of the naturally occurring proteins, where such variations are homologous or substantially similar to the naturally occurring protein, as described in greater detail below, be the naturally occurring protein the human protein or a protein from some other species that naturally expresses repressor protein, usually a mammalian species. In the following description of the subject invention, the name for a given repressor protein is used to refer not only to the human form of the protein, but also to homologs thereof expressed in non-human species, e.g., murine, rat, monkey and other mammalian species.

The subject repressor proteins are characterized by having TERT repressor activity. Specifically, the subject proteins bind to a repressor binding site present in the TERT minimal promoter. More specifically, the subject proteins bind to a “Site C” repressor binding site present in the human TERT minimal promoter. The human TERT Site C binding site is located in the region −80 to −50, and particularly −69 to −57 of the human TERT minimal promoter, and the subject repressor proteins bind to this site, where the sequence of this site is: GGCGCGAGTTTCA (SEQ ID NO:01). When binding to this site, or a portion thereof, the subject repressor proteins inhibit expression of TERT, where by inhibit expression is meant that expression of TERT is reduced by at least about 50%, usually at least about 75% and more usually at least about 90% as compared to a control system where TERT expression occurs and that is identical but for the absence of the subject repressor protein. The subject repressor proteins may be glycosylated, or modified in alternative ways.

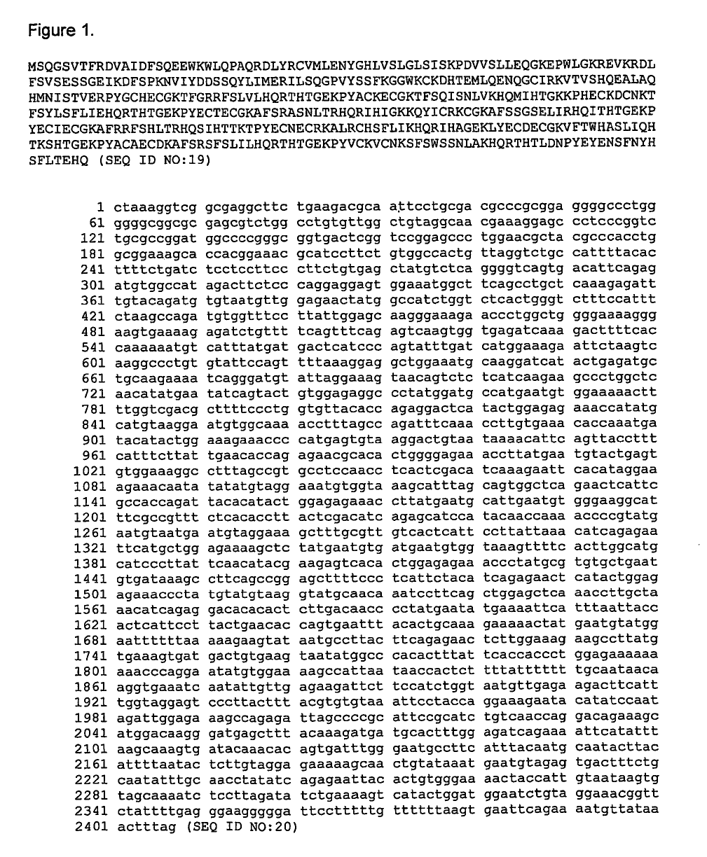

Of particular interest in certain embodiments as the telomerase repressor protein is a protein that has an amino acid sequence that includes a sequence that is substantially the same as, or identical to, the sequence of the human ZNF140 protein, whose sequence is provided in GENBANK accession no. NM_{—}003440 and described in FEBS Lett. Aug. 7, 1995; 369 (2-3): 153-7, Genomics 27 (2), 259-264 (1995). The amino acid (SEQ ID NO:19) and nucleotide (SEQ ID NO:20) sequences of the human ZNF140 protein is also provided in FIG. 1.

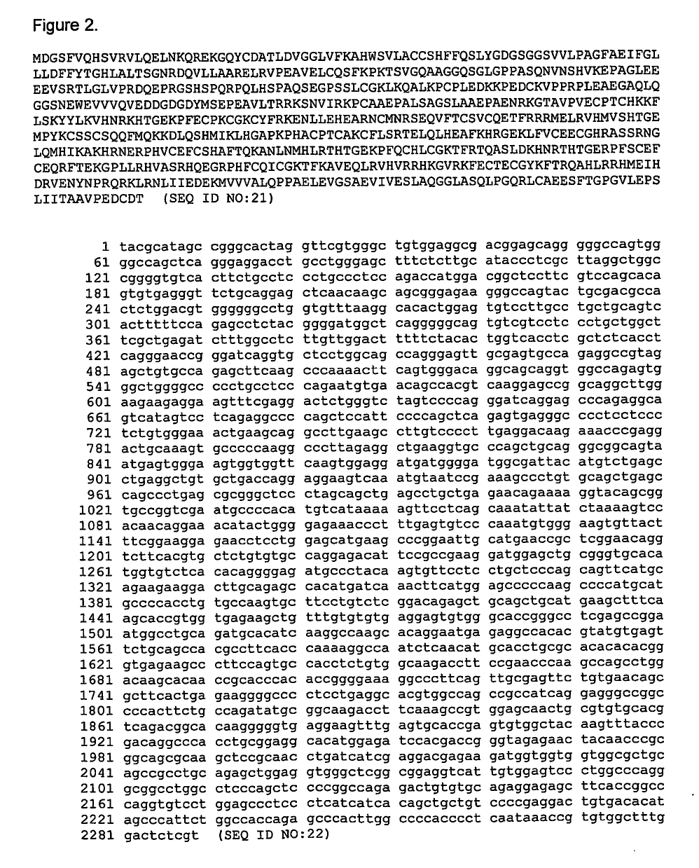

Of particular interest in yet other embodiments as the telomerase repressor protein is a protein that has an amino acid sequence that includes a sequence that is substantially the same as, or identical to, the sequence of the human HKR3 protein, whose sequence is provided in GENBANK accession nos. U45325 and NM_{—}005341 and described in Maris J. M. et al., Genomics 15; 35 (2): 289-298, 1996, White, P. S. et al., Eur J. Cancer 33 (12):1957-1961, 1997. The amino acid (SEQ ID NO:21) and nucleotide (SEQ ID NO:22) sequences of human HKR3 protein is also provided in FIG. 2.

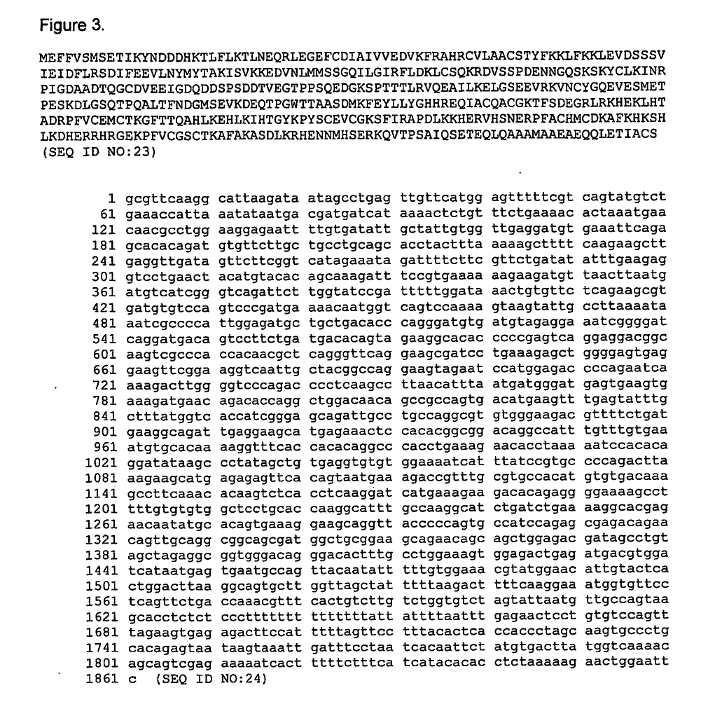

Of particular interest in yet other embodiments as the telomerase repressor protein is a protein that has an amino acid sequence that includes a sequence that is substantially the same as, or identical to, the sequence of the human ZFP161, whose sequence is provided in GENBANK accession nos. NM_{—}003409 and D89859 and further described in: Sugiura K. et al., Biochim. Biophys. Acta 1352 (1): 23-26, 1997, Sobe-Klocke, I. et al., Genomics 43(2):156-164, 1997. The amino acid (SEQ ID NO:23) and nucleotide (SEQ ID NO:24) sequences of human ZFP161 is also provided in FIG. 3.

By “substantially the same as” is meant a protein having a sequence that has at least about 50%, usually at least about 60% and more usually at least about 75%, and in many embodiments at least about 80%, usually at least about 90% and more usually at least about 95%, 96%, 97%, 98% or 99% sequence identity with the sequence of the above provided sequences, as measured by the BLAST compare two sequences program available on the NCBI website using default settings.

In addition to the specific TERT repressor proteins described above, homologs or proteins (or fragments thereof) from other species, i.e., other animal species, are also provided, where such homologs or proteins may be from a variety of different types of species, usually mammals, e.g., rodents, such as mice, rats; domestic animals, e.g. horse, cow, dog, cat; and primates, e.g., monkeys, baboons, humans etc. By homolog is meant a protein having at least about 35%, usually at least about 40% and more usually at least about 60% amino acid sequence identity to the specific human transcription repressor factors as identified above, where sequence identity is determined using the algorithm described supra.

The TERT repressor proteins of the subject invention are present in a non-naturally occurring environment, e.g., are separated from their naturally occurring environment. In certain embodiments, the subject proteins are present in a composition that is enriched for the subject proteins as compared to the subject proteins in their naturally occurring environment. As such, purified repressor proteins according to the subject invention are provided, where by purified is meant that the proteins are present in a composition that is substantially free of non repressor proteins of the subject invention, where by substantially free is meant that less than 90%, usually less than 60% and more usually less than 50% of the composition is made up of non-repressor proteins of the subject invention.

In certain embodiments of interest, the repressor proteins are present in a composition that is substantially free of the constituents that are present in its naturally occurring environment. For example, a human repressor protein comprising composition according to the subject invention in this embodiment will be substantially, if not completely, free of those other biological constituents, such as proteins, carbohydrates, lipids, etc., with which it is present in its natural environment. As such, protein compositions of these embodiments will necessarily differ from those that are prepared by purifying the protein from a naturally occurring source, where at least trace amounts of the constituents or other components of the protein's naturally occurring source will still be present in the composition prepared from the naturally occurring source.

The repressor proteins of the subject invention may also be present as isolates, by which is meant that the proteins are substantially free of both non-repressor proteins and other naturally occurring biologic molecules, such as oligosaccharides, polynucleotides and fragments thereof, and the like, where substantially free in this instance means that less than 70%, usually less than 60% and more usually less than 50% (by dry weight) of the composition containing the isolated repressor proteins is a non-repressor protein naturally occurring biological molecule. In certain embodiments, the repressor proteins are present in substantially pure form, where by substantially pure form is meant at least 95%, usually at least 97% and more usually at least 99% pure.

In addition to the naturally occurring proteins, polypepbdes that vary from the naturally occurring proteins are also provided. By polypeptide is meant proteins having an amino acid sequence encoded by an open reading frame (ORF) of a repressor protein gene, described below, including the full length protein and fragments thereof, particularly biologically active fragments and/or fragments corresponding to functional domains, and including fusions of the subject polypeptides to other proteins or parts thereof, e.g., immunoglobulin domains. Fragments of interest will typically be at least about 10 aa in length, usually at least about 50 aa in length, and may be as long as 300 aa in length or longer, but will usually not exceed about 1000 aa in length.

Also provided by the subject invention are ligands having TERT Site C binding activity. The term ligand, as used herein, refers to any compound capable of binding to a TERT repressor site, particularly Site C, and as such includes proteins and peptides, oligosaccharides, and the like, as well as binding mimetics thereof, including small molecule binding mimetics thereof. The subject ligands are capable of binding to Site C in a manner analogous to the binding activity of the subject repressor proteins, and will generally comprise the functional TERT binding domain of a repressor protein according to the subject invention, or the functional equivalent thereof.

Nucleic Acid Compositions

Also provided are nucleic acid compositions that encode TERT expression repressor polypeptides and fragments thereof, etc., as described above. Specifically, nucleic acid compositions encoding the subject polypeptides, as well as fragments or homologs thereof, are provided. By “nucleic acid composition” is meant a composition comprising a sequence of nucleotide bases that encodes a polypeptide according to the subject invention, i.e., a region of genomic DNA capable of being transcribed into mRNA that encodes a repressor polypeptide, the mRNA that encodes and directs the synthesis of a repressor polypeptide, etc. Also encompassed in this term are nucleic acids that are homologous, substantially similar or identical to the nucleic acids specifically disclosed herein.

Also provided are nucleic acids that are homologous to the provided nucleic acids, at least with respect to the coding regions thereof. The source of homologous nucleic acids to those specifically listed above may be any mammalian species, e.g., primate species, particularly human; rodents, such as rats and mice, canines, felines, bovines, equines, etc; as well as non-mammalian species, e.g., yeast, nematodes, etc. Between mammalian species, e.g., human and mouse, homologs have substantial sequence similarity, e.g., at least 75% sequence identity, usually at least 90%, more usually at least 95% between nucleotide sequences. Sequence similarity is calculated based on a reference sequence, which may be a subset of a larger sequence; such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Unless indicated otherwise, the sequence similarity values reported herein are those determined using the above referenced BLAST program using default settings. The sequences provided herein are essential for recognizing TERT repressor related and homologous polynucleotides in database searches. Of particular interest in certain embodiments are nucleic acids including a sequence substantially similar to the specific nucleic acids identified above, where by substantially similar is meant having sequence identity to this sequence of at least about 90%, usually at least about 95% and more usually at least about 99%.

Also provided are nucleic acids that hybridize to the above described nucleic acids under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

Nucleic acids encoding the proteins and polypeptides of the subject invention may be cDNAs or genomic DNAs, as well as fragments thereof. The nucleic acids may also be mRNAs, e.g., transcribed from genomic DNA, that encode (i.e. are translated into) the subject proteins and polypeptides. Also provided are genes encoding the subject proteins, where the term “gene” means the open reading frame encoding specific proteins and polypeptides, and introns that are present in the open reading frame, as well as adjacent 5′ and 3′ non-coding nucleotide sequences involved, e.g., untranslated regions, promoter or other regulatory elements, etc., in the regulation of expression, up to about 20 kb beyond the coding region, but possibly further in either direction. The gene may be introduced into an appropriate vector for extrachromosomal maintenance or for integration into a host genome.

The term “cDNA” as used herein is intended to include all nucleic acids that share the arrangement of sequence elements found in native mature mRNA species, where sequence elements at least include exons. Normally mRNA species have contiguous exons, with the intervening introns, when present, being removed by nuclear RNA splicing, to create a continuous open reading frame encoding a repressor protein according to the subject invention.

A genomic sequence of interest comprises the nucleic acid present between the initiation codon and the stop codon, as defined in the listed sequences, including all of the introns that are normally present in a native chromosome. It may further include specific transcriptional and translational regulatory sequences, such as promoters, enhancers, etc., including about 1 kb, but possibly more, of flanking genomic DNA at either the 5′ and 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acid compositions of the subject invention may encode all or a part of the subject proteins and polypeptides, described in greater detail above. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt.

The TERT repressor genes of the subject invention are isolated and obtained in substantial purity, generally as other than an intact chromosome. Usually, the DNA will be obtained substantially free of other nucleic acid sequences that do not include a TERT repressor protein sequence or fragment thereof, generally being at least about 50%, usually at least about 90% pure and are typically “recombinant,” i.e. flanked by one or more nucleotides with which it is not normally associated on a naturally occurring chromosome.

In addition to the plurality of uses described in greater detail in following sections, the subject nucleic acid compositions find use in the preparation of all or a portion of the subject polypeptides, as described above.

Also provided are nucleic acid probes, as well as constructs, e.g., vectors, expression systems, etc., as described more fully below, that include a nucleic acid sequence as described above. Probes of the subject invention are generally fragments of the provided nucleic acid. The probes may be a large or small fragment, generally ranging in length from about 10 to 100 nt, usually from about 15 to 50 nt. In using the subject probes, nucleic acids having sequence similarity are detected by hybridization under low stringency conditions, for example, at 50° C. and 6×SSC (0.9 M sodium chloride/0.09 M sodium citrate)(or analogous conditions) and remain bound when subjected to washing at higher stringency conditions, e.g., 55° C. in 1×SSC (0.15 M sodium chloride/0.015 M sodium citrate) (or analogous conditions). Sequence identity may be determined by hybridization under stringent conditions, for example, at 50° C. or higher and 0.1×SSC (15 mM sodium chloride/01.5 mM sodium citrate)(or analogous conditions). Nucleic acids having a region of substantial identity to the provided nucleic acid sequences bind to the provided sequences under stringent hybridization conditions. By using probes, particularly labeled probes of DNA sequences, one can isolate homologous or related sequences.

The subject nucleic acids are isolated and obtained in substantial purity, generally as other than an intact chromosome. As such, they are present in other than their naturally occurring environment. Usually, the DNA will be obtained substantially free of other nucleic acid sequences that do not include a repressor sequence or fragment thereof, generally being at least about 50%, usually at least about 90% pure and are typically “recombinant”, i.e. flanked by one or more nucleotides with which it is not normally associated on a naturally occurring chromosome.

The subject nucleic acids may be produced using any convenient protocol, including synthetic protocols, e.g., such as those where the nucleic acid is synthesized by a sequential monomeric approach (e.g., via phosphoramidite chemistry); where subparts of the nucleic acid are so synthesized and then assembled or concatamerized into the final nucleic acid, and the like. Where the nucleic acid of interest has a sequence that occurs in nature, the nucleic acid may be retrieved, isolated, amplified etc., from a natural source using conventional molecular biology protocols.

Also provided are constructs comprising the subject nucleic acid compositions, e.g., those that include a repressor protein coding sequence, inserted into a vector, where such constructs may be used for a number of different applications, including propagation, screening, genome alteration, and the like, as described in greater detail below. Constructs made up of viral and non-viral vector sequences may be prepared and used, including plasmids, as desired. The choice of vector will depend on the particular application in which the nucleic acid is to be employed. Certain vectors are useful for amplifying and making large amounts of the desired DNA sequence. Other vectors are suitable for expression in cells in culture, e.g., for use in screening assays. Still other vectors are suitable for transfer and expression in cells in a whole animal or person. The choice of appropriate vector is well within the ability of those of ordinary skill in the art. Many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo. Typically, homologous recombination is accomplished by attaching regions of homology to the vector on the flanks of the desired nucleotide sequence. Regions of homology are added by ligation of oligonucleotides, or by polymerase chain reaction using primers that include both the region of homology and a portion of the desired nucleotide sequence, for example.

Also provided are expression cassettes that include a coding sequence. By expression cassette is meant a nucleic acid that includes a sequence encoding a subject peptide or protein operably linked to a promoter sequence, where by operably linked is meant that expression of the coding sequence is under the control of the promoter sequence.

Preparation of Polypeptides According to the Subject Invention

The subject proteins may be obtained using any convenient protocol. As such, they may be obtained from naturally occurring sources or recombinantly produced. Naturally occurring sources of the subject proteins include tissues and portions/fractions, including cells and fractions thereof, e.g., extracts, homogenates etc., that include cells in which the desired protein is expressed.

The subject proteins may also be obtained from synthetic protocols, e.g., by expressing a recombinant gene encoding the subject protein, such as the polynucleotide compositions described above, in a suitable host under conditions sufficient for post-translational modification to occur in a manner that provides the expressed protein with TERT repression activity, e.g., Site C binding activity. For expression, an expression cassette may be employed. The expression cassette or vector will provide a transcriptional and translational initiation region, which may be inducible or constitutive, where the coding region is operably linked under the transcriptional control of the transcriptional initiation region, and under the translational control of the translational initiation region, and a transcriptional and translational termination region. These control regions may be native to a gene of the subject invention, or may be derived from exogenous sources.

Expression cassettes may be prepared comprising a transcription initiation region, the nucleic acid coding sequence or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the coding sequence. After introduction of the DNA, the cells containing the construct may be selected by means of a selectable marker, the cells expanded and then used for expression.

The subject proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the encoded protein will benefit from native folding and post-translational modifications. Small peptides can also be synthesized in the laboratory. Polypeptides that are subsets of the complete sequence may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433, DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilbum et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Geneic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci: (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980). 102:255, U.S. Pat. Nos. 4,767,704, 4,657,866, 4,927,762, 4,560,655, WO 90/103430, WO 87/00195, and U.S. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism.

Once the source of the protein is identified and/or prepared, e.g. a transfected host expressing the protein is prepared, the protein is then purified to produce the desired repressor protein comprising composition. Any convenient protein purification procedures may be employed, where suitable protein purification methodologies are described in Guide to Protein Purification, (Deuthser ed.) (Academic Press, 1990). For example, a lysate may be prepared from the original source, e.g. naturally occurring cells or tissues that express the subject repressor proteins or the expression host expressing the subject repressor proteins, and purified using HPLC, exclusion chromatography, gel electrophoresis, affinity chromatography, and the like.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670; the disclosure of which is herein incorporated by reference.

Antibodies

Also provided are antibodies that bind to the subject proteins and homologs thereof. Suitable antibodies are obtained by immunizing a host animal with peptides comprising all or a portion of the repressor protein. Suitable host animals include rat, sheep, goat, hamster, rabbit, etc. The origin of the protein immunogen may be mouse, rat, monkey etc. The host animal will generally be a different species than the immunogen, e.g. human protein used to immunize rabbit, etc.

The immunogen may comprise the complete protein, or fragments and derivatives thereof. Preferred immunogens comprise all or a part of the subject repressor protein, where these residues contain the post-translation modifications, such as glycosylation, found on the native target protein. Immunogens comprising the extracellular domain are produced in a variety of ways known in the art, e.g. expression of cloned genes using conventional recombinant methods, isolation from HEC, etc.

For preparation of polyclonal antibodies, the first step is immunization of the host animal with the target protein, where the target protein will preferably be in substantially pure form, comprising less than about 1% contaminant. The immunogen may include the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies of the subject invention may be produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using MPTS bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the-variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Methods of Modulating TRET Expression

Also provided by the subject invention are methods of modulating, including enhancing and repressing, TERT expression. As such, methods of both increasing and decreasing TERT expression are provided. In many embodiments, such methods are methods of modulating the binding of the repressor protein to a Site C site in a minimal TERT promoter, including enhancing or inhibiting binding of repressor protein to a TERT minimal promoter Site C site.

Enhancing TERT Expression

As such, methods are provided for enhancing TERT expression. By enhancing TERT expression is meant that the expression level of the TERT coding sequence is increased by at least about 2 fold, usually by at least about 5 fold and sometimes by at least about 25, about 50, or about 100 fold and in particular about 300 fold or higher, as compared to a control, i.e., expression from an expression system that is not subjected to the methods of the present invention. Alternatively, in cases where expression of the TERT gene is so low that it is undetectable, expression of the TERT gene is considered to be enhanced if expression is increased to a level that is easily detectable.

In these methods, repression of TERT expression is inhibited. By inhibited is meant that the repressive activity of the TERT site C repressor binding site/repressor interaction with respect to TERT expression is decreased by a factor sufficient to at least provide for the desired enhanced level of TERT expression, as described above. Inhibition of transcription repression by a repressor may be accomplished in a number of ways, where representative protocols for inhibiting the repression are now provided.

One representative method of inhibiting repression of transcription is to employ double-stranded, i.e., duplex, oligonucleotide decoys for a repressor protein, which bind to the repressor protein thereby preventing repressor protein binding to its target site in the TERT promoter, e.g., the Site C site of the TERT minimal promoter. These duplex oligonucleotide decoys will have at least that portion of the sequence of the TERT Site C site, e.g., as encoded by a nucleic acid having a sequence of SEQ ID NO:01, or a nucleic acid having a sequence substantially similar to identical thereto, as described above. In many embodiments, the length of these duplex oligonucleotide decoys ranges from about 5 to 5000, usually from about 5 to 500 and more usually from about 10 to 50 bases. In using such oligonucleotide decoys, the decoys are placed into the environment of the expression system and a target repressor protein, resulting in de-repression of the transcription and expression of the TERT coding sequence. Oligonucleotide decoys and methods for their use and administration are further described in general terms in Morishita et al., Circ Res (1998) 82 (10):1023-8. These oligonucleotide decoys generally include a TERT site C repressor binding site recognized by the target repressor protein, including the specific regions detailed above, where these particular embodiments are nucleic acid compositions of the subject invention, as defined above.

Instead of the above described decoys, other agents that disrupt binding of the repressor protein to the target TERT Site C repressor binding site and thereby inhibit repression may be employed. Other agents of interest include, among other types of agents, small molecules that bind to the repressor and inhibit its binding the Site C repressor region. Alternatively, agents that bind to the Site C sequence and inhibit its binding to the repressor are of interest. Alternatively, agents that disrupt protein-protein interactions with cofactors, e.g., cofactor binding, and thereby inhibit repression are of interest. Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below. Small molecule agents of particular interest include pyrrole-imidazole polyamides, analogous to those described in Dickinson et al., Biochemistry Aug. 17, 1999;38(33):10801-7. Other agents include “designer” DNA binding proteins that bind Site C (without causing repression) and prevent the repressor protein from binding.

In yet other embodiments, expression of the target repressor protein is inhibited. Inhibition of target repressor protein expression may be accomplished using any convenient means, including administration of an agent that inhibits target repressor protein expression (e.g., antisense agents), inactivation of the encoding gene, e.g., through recombinant techniques, etc.

Antisense molecules can be used to down-regulate expression of the target protein in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3N—ON-5N—S-phosphorothioate, 3N—S-5N—O-phosphorothioate, 3N—CH₂-5N—O-phosphonate and 3N—NH-5N—O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2N—OH of the ribose sugar may be altered to form 2N—O-methyl or 2N—O-allyl sugars, which provides resistance to degradation without comprising affinity. Modification of the heterocyclic bases must maintain proper base pairing. Some useful substitutions include deoxyuridine for deoxythymidine; 5-methyl-2N-deoxycytidine and 5-bromo-2N-deoxycytidine for deoxycytidine. 5-propynyl-2N-deoxyuridine and 5-propynyl-2N-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a met al complex, e.g. terpyridylCu(II), capable of mediating mRNA hydrolysis are described in Bashkin et al. (1995), Appl. Biochem. Biotechnol. 54:43-56.

In another embodiment, the target repressor protein gene is inactivated so that it no longer expresses the target repressor protein. By inactivated is meant that the gene, e.g., coding sequence and/or regulatory elements thereof, is genetically modified so that it no longer expresses a protein, or at least a functional protein. The alteration or mutation may take a number of different forms, e.g., through deletion of one or more nucleotide residues in the repressor region, through exchange of one or more nucleotide residues in the repressor region, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

The above described methods of enhancing TERT expression find use in a number of different applications. In many applications, the subject methods and compositions are employed to enhance TERT expression in a cell that endogenously comprises a TERT gene, e.g., for enhancing expression of hTERT in a normal human cell in which TERT expression is repressed. The target cell of these applications is, in many instances, a normal cell, e.g. a somatic cell. Expression of the TERT gene is considered to be enhanced if, consistent with the above description, in those cells that detectably express TERT, expression is increased by at least about 2 fold, usually at least about 5 fold and often by at least about 25, about 50, about 100 fold or higher, as compared to a control, e.g., an otherwise identical cell not subjected to the subject methods, or becomes detectable from an initially undetectable state, as described above. Alternatively, in those cells that initially do not detectably express TERT, TERT expression is enhanced to at least a detectable level.

A more specific application in which the subject methods find use is to increase the proliferative capacity of a cell. The term “proliferative capacity” as used herein refers to the number of divisions that a cell can undergo, and preferably to the ability of the target cell to continue to divide where the daughter cells of such divisions are not transformed, i.e., they maintain normal response to growth and cell cycle regulation. The subject methods typically result in an increase in proliferative capacity of at least about 1.2-2 fold, usually at least about 5 fold and often at least about 10, 20, 50 fold or even higher, compared to a control. As such, yet another more specific application in which the subject methods find use is in the delay of the occurrence of cellular senescence. By practicing the subject methods, the onset of cellular senescence may be delayed by a factor of at least about 1.2-2 fold, usually at least about 5 fold and often at least about 10, 20, 50 fold or even higher, compared to a control.

Methods of Inhibiting TERT Expression

As mentioned above, also provided are methods for enhancing repressor protein mediated repression of TERT expression, and thereby inhibiting TERT expression. In such methods, the amount and/or activity of the target repressor protein is increased so as to enhance its repression of TERT expression. A variety of different protocols may be employed to achieve this result, including administration of an effective amount of the repressor protein or analog/mimetic thereof, an agent that enhances expression of, or an agent that enhances the activity of, the repressor protein.

As such, the nucleic acid compositions of the subject invention find use in situations where one wishes to enhance a repressor protein activity in a host The genes, gene fragments, or the encoded proteins or protein fragments are useful in gene therapy to treat disorders in which inhibition of TERT expression is desired, including those applications described in greater detail below. Expression vectors may be used to introduce the gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjecton, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

Therapeutic Applications of TERT Expression Modulation

The methods also find use in a variety of therapeutic applications in which it is desired to modulate, e.g., increase or decrease, TERT expression in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy. In such methods, an effective amount of an active agent that modulates TERT expression, e.g., enhances or decreases TERT expression as desired, is administered to the target cell or cells, e.g., by contacting the cells with the agent, by administering the agent to the animal, etc. By effective amount is meant a dosage sufficient to modulate TERT expression in the target cell(s), as desired.

In the subject methods, the active agent(s) may be administered to the targeted cells using any convenient means capable of resulting in the desired enhancement of TERT expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be:made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agent, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

The subject methods find use in the treatment of a variety of different conditions in which the modulation, e.g., enhancement or inhibition, of TERT expression in the host is desired. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

As indicated above, the subject invention provides methods of treating disease conditions resulting from a lack of TERT expression and methods of treating disease conditions resulting from unwanted TERT expression. Representative disease conditions for each category are now described in greater detail separately.

Treatment of Disease Conditions by Increasing TERT Expression

One representative disease condition that may be treated according to the subject invention is Progeria, or Hutchinson-Gilford syndrome. This condition is a disease of shortened telomeres for which no known cure exists. It afflicts children, who seldom live past their early twenties. In many ways progeria parallels aging itself. However, these children are born with short telomeres. Their telomeres don't shorten at a faster rate; they are just short to begin with. The subject methods can be used in such conditions to further delay natural telomeric shortening and/or increase telomeric length, thereby treating this condition.

Another specific disease condition in which the subject methods find use is in immune senescence. The effectiveness of the immune system decreases with age. Part of this decline is due to fewer T-lymphocytes in the system, a result of lost replicative capacity. Many of the remaining T-lymphocytes experience loss of function as their telomeres shorten and they approach senescence. The subject methods can be employed to inhibit immune senescence due to telomere loss. Because hosts with aging immune systems are at greater risk of developing pneumonia, cellulitis, influenza, and many other infections, the subject methods reduce morbidity and mortality due to infections.

The subject methods also find use in AIDS therapy. HIV, the virus that causes AIDS, invades white blood cells, particularly CD4 lymphocyte cells, and causes them to reproduce high numbers of the HIV virus, ultimately killing cells. In response to the loss of immune cells (typically about a billion per day), the body produces more CD8 cells to be able to suppress infection. This rapid cell division accelerates telomere shortening, ultimately hastening immune senescence of the CD8 cells. Anti-retroviral therapies have successfully restored the immune systems of AIDS patients, but survival depends upon the remaining fraction of the patient's aged T-cells. Once shortened, telomere length has not been naturally restored within cells. The subject methods can be employed to restore this length and/or prevent further shortening. As such the subject methods can spare telomeres and is useful in conjunction with the anti-retroviral treatments currently available for HIV.

Yet another type of disease condition in which the subject methods find use is cardiovascular disease. The subject methods can be employed to extend telomere length and replicative capacity of endothelial cells lining blood vessel walls (DeBono, Heart 80:110-1, 1998). Endothelial cells form the inner lining of blood vessels and divide and replace themselves in response to stress. Stresses include high blood pressure, excess cholesterol, inflammation, and flow stresses at forks in vessels. As endothelial cells age and can no longer divide sufficiently to replace lost cells, areas under the endothelial layer become exposed. Exposure of the underlying vessel wall increases inflammation, the growth of smooth muscle cells, and the deposition of cholesterol. As a result, the vessel narrows and becomes scarred and irregular, which contributes to even more stress on the vessel (Cooper, Cooke and Dzau, J Gerontol Biol Sci 49: 191-6, 1994). Aging endothelial cells also produce altered amounts of trophic factors (hormones that affect the activity of neighboring cells). These too contribute to increased clotting, proliferation of smooth muscle cells, invasion by white blood cells, accumulation of cholesterol,.and other changes, many of which lead to plaque formation and clinical cardiovascular disease (Ibid.). By extending endothelial cell telomeres, the subject methods can be employed to combat the stresses contributing to vessel disease. Many heart attacks may be prevented if endothelial cells were enabled to continue to divide normally and better maintain cardiac vessels. The occurrence of strokes caused by the aging of brain blood vessels may also be significantly reduced by employing the subject methods to help endothelial cells in the brain blood vessels to continue to divide and perform their intended function.

The subject methods also find use in skin rejuvenation. The skin is the first line of defense of the immune system and shows the most visible signs of aging (West, Arch Dermatol 130(1):87-95, 1994). As skin ages, it thins, develops wrinkles, discolors, and heals poorly. Skin cells divide quickly in response to stress and trauma; but, over time, there are fewer and fewer actively dividing skin cells. Compounding the loss of replicative capacity in aging skin is a corresponding loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells have shorter telomeres and are less capable of dividing (Fossel, REVERSING HUMAN AGING. William Morrow & Company, New York City, 1996).

By practicing the subject methods, e.g., via administration of an active agent topically, one can extend telomere length, and slow the downward spiral that skin experiences with age. Such a product not only helps protect a person against the impairments of aging skin; it also permits rejuvenated skin cells to restore youthful immune resistance and appearance. The subject methods can be used for both medical and cosmetic skin rejuvenation applications.

Yet another disease condition in which the subject methods find use in the treatment of osteoporosis. Two types of cells interplay in osteoporosis: osteoblasts make bone and osteoclasts destroy it. Normally, the two are in balance and maintain a constant turnover of highly structured bone. In youth, bones are resilient, harder to break, and heal quickly. In old age, bones are brittle, break easily, and heal slowly and often improperly. Bone loss has been postulated to occur because aged osteoblasts, having lost much of their replicative capacity, cannot continue to divide at the rate necessary to maintain balance (Hazzard et al. PRINCIPLES OF GERIATRIC MEDICINE AND GERONTOLOGY, 2d ed. McGraw-Hill, New York City, 1994). The subject methods can be employed to lengthen telomeres of osteoblast and osteoclast stem cells, thereby encouraging bone replacement and proper remodeling and reinforcement. The resultant stronger bone improves the quality of life for the many sufferers of osteoporosis and provides savings from fewer fracture treatments. The subject methods are generally part of a comprehensive treatment regime that also includes calcium, estrogen, and exercise.

Additional disease conditions in which the subject methods find use are described in WO 99/35243, the disclosures of which are herein incorporated by reference.

In addition to the above described methods, the subject methods can also be used to extend the lifetime of a mammal. By extend the lifetime is meant to increase the time during which the animal is alive, where the increase is generally at least 1%, usually at least 5% and more usually at least about 10%, as compared to a control.

As indicated above, instead of a multicellular animal, the target may be a cell or population of cells which are treated according to the subject methods and then introduced into a multicellular organism for therapeutic effect. For example, the subject methods may be employed in bone marrow transplants for the treatment of cancer and skin grafts for bum victims. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum Gene Ther 7(11): 1347-50, 1996). In such cases, the therapeutic cells must be expanded from a single engineered cell. By the time there are sufficient cells for transplantation, the cells have undergone the equivalent of 50 years of aging (Decary, Mouly et al. Hum Gene Ther 8(12): 1429-38,1997). Use of the subject methods spares the replicative capacity of bone marrow cells and skin cells during culturing and expansion and thus significantly improves the survival and effectiveness of bone marrow and skin cell transplants. Any transplantation technology requiring cell culturing can benefit from the subject methods, including ex vivo gene therapy applications in which cells are cultured outside of the animal and then administered to the animal, as described in U.S. Pat. Nos. 6,068,837; 6,027,488; 5,824,655; 5,821,235; 5,770,580; 5,756,283; 5,665,350; the disclosures of which are herein incorporated by reference.

Treatment of Disease Conditions by Decreasing TERT Expression

As summarized above, also provided are methods for enhancing repression of TERT expression, where by enhancement of TERT expression repression is meant a decrease in TERT expression by a factor of at least about 2 fold, usually at least about 5 fold and more usually at least about 10 fold, as compared to a control. Methods for enhancing repressor protein mediated TERT expression repression find use in, among other applications, the treatment of cellular proliferative disease conditions, including neoplastic disease conditions, i.e., cancer. In such applications, an effective amount of an active agent, e.g., a repressor protein, analog or mimetic thereof, a vector encoding a repressor protein or active fragment thereof, an agent that enhances endogenous repressor protein activity, an agent that enhances expression of a repressor protein, etc., is administered to the subject in need thereof. Treatment is used broadly as defined above, e.g., to include at least an amelioration in one or more of the symptoms of the disease, as well as a complete cessation thereof, as well as a reversal and/or complete removal of the disease condition, e.g., cure. Methods of treating disease conditions resulting from unwanted TERT expression, such as cancer and other diseases characterized by the presence of unwanted cellular proliferation, are described in, for example, U.S. Pat. Nos. 5,645,986; 5,656,638; 5,703,116; 5,760,062; 5,767,278; 5,770,613; and 5,863,936; the disclosures of which are herein incorporated by reference.

Screening Assays

Also provided by the subject invention are screening protocols and assays for identifying agents that modulate, e.g., inhibit or enhance, repressor protein repression of TERT transcription. As such, the screening assays are assays that provide for the identification of agents that modulate, e.g., inhibit or enhance, the binding interaction between a repressor protein and Site C.

The screening methods will typically be assays which provide for qualitative/quantitative measurements of TERT promoter controlled expression, e.g., of a coding sequence for a marker or reporter gene, in the presence of a particular candidate therapeutic agent. For example, the assay could be an assay which measures the TERT promoter controlled expression of a reporter gene (i.e. coding sequence, e.g., luciferase, SEAP, etc.) in the presence and absence of a candidate inhibitor agent, e.g. the expression of the reporter gene in the presence or absence of a candidate agent. The screening method may be an in vitro or in vivo format, where both formats are readily developed by those of skill in the art. In other words, such assays can be done in vivo or in vitro in mammalian, non-mammalian, Yeast, bacteria, etc.

In Vitro Models of Repressor Protein Function

in vitro models of repressor protein function are provided. Of particular interest are models of repressor protein TERT binding events in which the TERT binding site is Site C. Such models typically include: a Site C site, a repressor protein polypeptide and a modulatory agent, e.g., competitor or inhibitor, which are present under conditions sufficient to inhibit repressor protein/site C binding. The competitor may be any compound that is, or is suspected to be, a compound capable of specifically binding to the repressor protein, where of particular interest in many embodiments is the use of the subject ligands described above as competitors. Depending on the particular model, one or more of, usually one of, the specified components may be labeled, where by labeled is meant that the components comprise a detectable moiety, e.g. a fluorescent or radioactive tag, or a member of a signal producing system; e.g. biotin for binding to an enzyme-streptavidin conjugate in which the enzyme is capable of converting a substrate to a chromogenic product.

The above in vitro models may be designed a number of different ways, where a variety of assay configurations and protocols may be employed, as are known in the art. For example, one of the components may be bound to a solid support, and the remaining components contacted with the support bound component. The above components of the method may be combined at substantially the same time or at different times, e.g. soluble repressor protein and a competitor ligand may be combined first, and the resultant mixture subsequently combined with bound site C sequence. Following the contact step, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound repressor protein/Site C complexes will then be detected.

In alternative in vitro models, an expression cassette including a reporter gene under control of a Site C sequence and repressor protein may be present in a cell free environment in which the reporter gene is expressed in the absence of repressor protein binding to the Site C region. By expression cassette or system is meant a nucleic acid that includes a sequence encoding a peptide or protein of interest, i.e., a coding sequence, operably linked to a promoter sequence, where by operably linked is meant that expression of the coding sequence is under the control of the promoter sequence. The expression systems and cassettes of the subject invention include a Site C repressor binding site/region operably linked to the promoter, where the promoter is, in many embodiments, a TERT promoter, such as the hTERT promoter. See e.g., the hTERT promoter sequence described in Cong et al., Hum. Mol. Genet. (1999) 8:137-142. The in vitro model further includes a coding sequence of interest operably linked to the Site C binding site. The expression system is then employed in an appropriate cell free environment that includes the repressor protein to provide expression or non-expression of the protein, as desired.

In Vivo Models of Repressor Protein Function

A variety of different in vivo models of repressor protein function are also provided by the subject invention and may be used in the screening assays of the subject invention. In vivo models of interest include engineered cells that include an expression cassette as described above and a repressor protein, which components are present in a host cell. Also of interest in the subject screening assays are multicellular in vivo models, e.g., the transgenic animal models described below.

Whether the format is in vivo or in vitro, the model being employed is combined with the candidate agent and the effect of the candidate agent on model is observed and related to the TERT expression modulatory activity of the agent. For example, for screening inhibitory agents, the model is combined with the candidate agent in an environment in which, in the absence of the candidate agent, the TERT promoter is repressed, e.g., in the presence of a repressor protein, that interacts with the TERT Site C repressor binding site and causes TERT promoter repression. The conditions may be set up in vitro by combining the various required components in an aqueous medium, or the assay may be carried out in vivo, etc.

Alternatively, the repressor protein could be engineered to replace the repressor domain with an activation domain (or other detectable domain), but still retaining the DNA binding domain. In this manner, assays can be set up in which agents that are candidates for preventing the repressor protein DNA binding domain from binding to the DNA binding site can be screened (as described in the above paragraph) for activation (or other signal) of the reporter gene instead of repression.

Likewise, the repressor protein could be engineered to replace the DNA binding domain with another DNA binding domain (e.g. p53), but still retaining the repression domain. In this manner, assays can be set up in which agents that are candidates for preventing the repression domain from binding to cofactors (protein-protein interaction) can be screened using DNA binding domains that have already been well characterized. In this manner, agents that enhance and inhibit protein-protein interactions with cofactors involved in TERT expression repression may be identified.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents identified in the above screening assays that inhibit repression of TERT transcription find use in the methods described above, e.g., in the enhancement of TERT expression. Alternatively, agents identified in the above screening assays that enhance repression find use in applications where inhibition of TERT expression is desired, e.g., in the treatment of disease conditions characterized by the presence of unwanted TERT expression, such as cancer and other diseases characterized by the presence of unwanted cellular proliferation, where such methods are described in, for example, U.S. Pat. Nos. 5,645,986; 5,656,638; 5,703,116; 5,760,062; 5,767,278; 5,770,613; and 5,863,936; the disclosures of which are herein incorporated by reference.

Generation of Antibodies

In one embodiment of the invention, the blocking of the repressor protein mediated TERT repression is used to immortalize cells in culture. Exemplary of cells that may be used for this purpose are non-transformed antibody producing cells, e.g. B cells and plasma cells which may be isolated and identified for their ability to produce a desired antibody using known technology as, for example, taught in U.S. Pat. No. 5,627,052. These cells may either secrete antibodies (antibody-secreting cells) or maintain antibodies on the surface of the cell without secretion into the cellular environment. Such cells have a limited lifespan in culture, and are usefully immortalized by upregulating expression of telomerase using the methods of the present invention.

Because the above described methods are methods of increasing expression of TERT and therefore increasing the proliferative capacity and/or delaying the onset of senescence in a cell, they find applications in the production of a range of reagents, typically cellular or animal reagents. For example, the subject methods may be employed to increase proliferation, delay senescence and/or extend the lifetimes of cultured cells. Cultured cell populations having enhanced TERT expression are produced using any of the protocols as described above, including by contact with an agent that inhibits repressor region transcription repression and/or modification of the repressor region in a manner such that it no longer represses TERT coding sequence transcription, etc.

The subject methods find use in the generation of monoclonal antibodies. An antibody-forming cell may be identified among antibody-forming cells obtained from an animal which has either been immunized with a selected substance, or which has developed an immune response to an antigen as a result of disease. Animals may be immunized with a selected antigen using any of the techniques well known in the art suitable for generating an immune response. Antigens may include any substance to which an antibody may be made, including, among others, proteins, carbohydrates, inorganic or organic molecules, and transition state analogs that resemble intermediates in an enzymatic process. Suitable antigens include, among others, biologically active proteins, hormones, cytokines, and their cell surface receptors, bacterial or parasitic cell membrane or purified components thereof, and vital antigens.

As will be appreciated by one of ordinary skill in the art, antigens which are of low immunogenicity may be accompanied with an adjuvant or hapten in order to increase the immune response (for example, complete or incomplete Freund's adjuvant) or with a carrier such as keyhole limpet hemocyanin (KLH).

Procedures for immunizing animals are well known in the art. Briefly, animals are injected with the selected antigen against which it is desired to raise antibodies. The selected antigen may be accompanied by an adjuvant or hapten, as discussed above, in order to further increase the immune response. Usually the substance is injected into the peritoneal cavity, beneath the skin, or into the muscles or bloodstream. The injection is repeated at varying intervals and the immune response is usually monitored by detecting antibodies in the serum using an appropriate assay that detects the properties of the desired antibody. Large numbers of antibody-forming cells can be found in the spleen and lymph node of the immunized animal. Thus, once an immune response has been generated, the animal is sacrificed, the spleen and lymph nodes are removed, and a single cell suspension is prepared using techniques well known in the art.

Antibody-forming cells may also be obtained from a subject which has generated the cells during the course of a selected disease. For instance, antibody-forming cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-forming cells may be obtained from subjects with disease due to known etiological agents such as malaria or AIDS. These antibody forming cells may be derived from the blood or lymph nodes, as well as from other diseased or normal tissues. Antibody-forming cells may be prepared from blood collected with an anticoagulant such as heparin or EDTA. The antibody-forming cells may be further separated from erythrocytes and polymorphs using standard procedures such as centrifugation with Ficoll-Hypaque (Pharmacia, Uppsula, Sweden). Antibody-forming cells may also be prepared from solid tissues such as lymph nodes or tumors by dissociation with enzymes such as collagenase and trypsin in the presence of EDTA.

Antibody-forming cells may also be obtained by culture techniques such as in vitro immunization. Briefly, a source of antibody-forming cells, such as a suspension of spleen or lymph node cells, or peripheral blood mononuclear cells are cultured in medium such as RPMI 1640 with 10% fet al bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL4, IL-5, IL-6, GM-CSF, and IFN-γ. To enhance immunogenicity, the selected antigen may be coupled to the surface of cells, for example, spleen cells, by conventional techniques such as the use of biotin/avidin as described below.

Antibody-forming cells may also be obtained from very early monoclonal or oligoclonal fusion cultures produced by conventional hybridoma technology. The present invention is advantageous in that it allows rapid selection of antibody-forming cells from unstable, interspecies hybridomas, e.g., formed by fusing antibody-forming cells from animals such as rabbits, humans, cows, pigs, cats, and dogs with a murine myeloma such NS-1.

Antibody-forming cells may be enriched by methods based upon the size or density of the antibody-forming cells relative to other cells. Gradients of varying density of solutions of bovine serum albumin can also be used to separate cells according to density. The fraction that is most enriched for desired antibody-forming cells can be determined in a preliminary procedure using the appropriate indicator system in order to establish the antibody-forming cells.

The identification and culture of antibody producing cells of interest is followed by enhancement of TERT expression is these cells by the subject methods, thereby avoiding the need for the immortalization/fusing step employed in traditional hybridoma manufacture protocols. In such methods, the first step is immunization of the host animal with an immunogen, typically a polypeptide, where the polypeptide will preferably be in substantially pure form, comprising less than about 1% contaminant. The immunogen may comprise the complete protein, fragments or derivatives thereof. To increase the immune response of the host animal, the protein may be combined with an adjuvant, where suitable adjuvants include alum, dextran, sulfate, large polymeric anions, oil & water emulsions, e.g. Freund's adjuvant, Freund's complete adjuvant, and the like. The protein may also be conjugated to synthetic carrier proteins or synthetic antigens. A variety of hosts may be immunized to produce the subject antibodies. Such hosts include rabbits, guinea pigs; rodents (e.g. mice, rats), sheep, goats, and the like. The protein is administered to the host, usually intradermally, with an initial dosage followed by one or more, usually at least two, additional booster dosages. Following immunization, generally, the spleen and/or lymph nodes of an immunized host animal provide a source of plasma cells. The plasma cells are treated according to the subject invention to enhance TERT expression and thereby, increase the proliferative capacity and/or delay senescence to produce “pseudo” immortalized cells. Culture supernatant from individual cells is then screened using standard techniques to identify those producing antibodies with the desired specificity. Suitable animals for production of monoclonal antibodies to a human protein include mouse, rat, hamster, etc. To raise antibodies against the mouse protein, the animal will generally be a hamster, guinea pig, rabbit, etc. The antibody may be purified from the cell supernatants or ascites fluid by conventional techniques, e.g. affinity chromatography using RFLAT-1 protein bound to an insoluble support, protein A sepharose, etc.

In an analogous fashion, the subject methods are employed to enhance TERT expression in non-human animals, e.g., non-human animals employed in laboratory research. Using the subject methods with such animals can provide a number of advantages, including extending the lifetime of difficult and/or expensive to produce transgenic animals. As with the above described cells and cultures hereof, the expression of TERT in the target animals may be enhanced using a number of different protocols, including the administration of an agent that inhibits SC repression and/or targeted disruption of the repressor binding site. The subject methods may be used with a number of different types of animals, where animals of particular interest include mammals, e.g., rodents such as mice and rats, cats, dogs, sheep, rabbits, pigs, cows, horses, and non-human primates, e.g. monkeys, baboons, etc.

Additional Utilities

The subject polypeptide and nucleic acid compositions find use in a variety of additional applications. Applications in which the subject polypeptide and nucleic acid compositions find use include: (a) the identification of homologs; (b) as a source of novel promoter elements; (c) the identification of expression regulatory factors; (d) as probes and primers in hybridization applications, e.g. PCR; (e) the identification of expression patterns in biological specimens; (f) the preparation of cell or animal models for function; (g) the preparation of in vitro models for function; (h) the identification of binding proteins; (i) the identification of binding DNA's (e.g. other promoters); etc.

Identification of Homologs

Homologs are identified by any of a number of methods. A fragment of the provided cDNA may be used as a hybridization probe against a cDNA library from the target organism of interest, where low stringency conditions are used. The probe may be a large fragment, or one or more short degenerate primers. Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/0.015 M sodium citrate). Sequence identity may be determined by hybridization under stringent conditions, for example, at 50° C. or higher and 0.1×SSC (15 mM sodium chloride/01.5 mM sodium citrate). Nucleic acids having a region of substantial identity to the provided sequences, e.g. allelic variants, genetically altered versions of the gene, etc., bind to the provided sequences under stringent hybridization conditions. By using probes, particularly labeled probes of DNA sequences, one can isolate homologous or related genes.

Identification of Novel Promoter Elements

The sequence of the 5′ flanking region may be utilized for promoter elements, including enhancer binding sites, that provide for regulation in tissues where the repressor protein is expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Identification of Expression Regulatory Factors

Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell et al. (1995), Mol. Med. 1:194-205; Mortlock et al. (1996), Genome Res. 6:327-33; and Joulin and Richard-Foy (1995), Eur. J. Biochem. 232:620-626.

The regulatory sequences may be used to identify cis acting sequences required for transcriptional or translational regulation of gene expression, especially in different tissues or stages of development, and to identify cis acting sequences and trans-acting factors that regulate or mediate gene expression. Such transcription or translational control regions may be operably linked to a repressor protein gene in order to promote expression of wild type or altered or other proteins of interest in cultured cells, or in embryonic, fetal or adult tissues, and for gene therapy.

Probes and Primers

Small DNA fragments are useful as primers for PCR, hybridization screening probes, etc. Larger DNA fragments, i.e. greater than 100 nt are useful for production of the encoded polypeptide, as described in the previous section. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

Identification of Expression Patterns in Biological Specimens

The DNA may also be used to identify expression of the gene in a biological specimen. The manner in which one probes cells for the presence of particular nucleotide sequences, as genomic DNA or RNA, is well established in the literature. Briefly, DNA or mRNA is isolated from a cell sample. The mRNA may be amplified by RT-PCR, using reverse transcriptase to form a complementary DNA strand; followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of gene expression in the sample.

The Preparation of Mutants

The sequence of a gene, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon. Other modifications of interest include epitope tagging, e.g. with the FLAG system, HA, etc. For studies of subcellular localization, fusion proteins with green fluorescent proteins (GFP) may be used.

Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:35-41; Sayers et al (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4. Such mutated genes may be used to study structure-function relationships, or to alter properties of the protein that affect its function or regulation.

Production of In Vivo Models of Repressor Protein Function

The subject nucleic acids can be used to generate transgenic, non-human animals or site specific gene modifications in cell lines. Transgenic animals may be made through homologous recombination, where the normal locus is altered. Alternatively, a nucleic acid construct is randomly integrated into the genome. Vectors for stable integration include plasmids, retroviruses and other animal viruses, YACs, and the like.

The modified cells or animals are useful in the study of repressor function and regulation. For example, a series of small deletions and/or substitutions may be made in the host's native gene to determine the role of different exons in the regulation of telomerase expression, e.g. repression of TERT promoter. Specific constructs of interest include ant-sense, which will block expression, expression of dominant negative mutations, and over-expression of repressor protein genes. Where a sequence is introduced, the introduced sequence may be either a complete or partial sequence of a gene native to the host, or may be a complete or partial sequence that is exogenous to the host animal, e.g., a human sequence. A detectable marker, such as lac Z, may be introduced into the locus, where upregulation of expression will result in an easily detected change in phenotype.

One may also provide for expression of the gene or variants thereof in cells or tissues where it is not normally expressed (e.g., Mammalian, non-Mammalian, Yeast, Bacterial, etc. cells), at levels not normally present in such cells or tissues, or at abnormal times of development. DNA constructs for homologous recombination will comprise at least a portion of the gene native to the species of the host animal, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc., e.g. to determine the effect of a candidate drug on repressor protein activity.

Production of In Vitro Models of Repressor Protein Function

Also provided by the subject invention are in vitro models of repressor protein function, e.g. the role of the repressor protein as a TERT repressor. Of particular interest is the modulation of repressor protein TERT binding events in which the TERT binding site is site C. In in vitro methods of inhibiting TERT repressor binding events, such methods typically include a competitor or inhibitor under conditions sufficient to inhibit Site C binding to occur. The competitor may be any compound that is, or is suspected to be, a compound capable of specifically binding to a repressor protein, where of particular interest in many embodiments is the use of the subject ligands described above as competitors. Depending on the particular method, one or more of, usually one of, the specified components may be labeled, where by labeled is meant that the components comprise a detectable moiety, e.g. a fluorescent or radioactive tag, or a member of a signal producing system, e.g. biotin for binding to an enzyme-streptavidin conjugate in which the enzyme is capable of converting a substrate to a chromogenic product.

The above in vitro methods may be designed a number of different ways, where a variety of assay configurations and protocols may be employed, as are known in the art. For example, one of the components may be bound to a solid support, and the remaining components contacted with the support bound component. The above components of the method may be combined at substantially the same time or at different times, e.g. soluble repressor protein and a competitor ligand may be combined first, and the resultant mixture subsequently combined with bound Site C sequence. Following the contact step, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound Site C complexes will then be detected.

The above described in vitro methods find use in screening assays designed to identify the presence or absence of repressor protein in cancerous cells. The above described in vitro methods also find use in screening assays designed to identify compounds that inhibit the binding of repressor protein to the TERT promoter.

Diagnostic Applications

Also provided are methods of diagnosing disease states associated with repressor protein activity or the absence thereof, e.g., based on observed levels of repressor protein or the expression level of the gene in a biological sample of interest. Samples, as used herein, include biological fluids such as blood, cerebrospinal fluid, tears, saliva, lymph, dialysis fluid and the like; organ or tissue culture derived fluids; and fluids extracted from physiological tissues. Also included in the term are derivatives and fractions of such fluids. The cells may be dissociated; in the case of solid tissues, or tissue sections may be analyzed. Alternatively a lysate of the cells may be prepared.

A number of methods are available for determining the expression level of a gene or protein in a particular sample. Diagnosis may be performed by a number of methods to determine the absence or presence or altered amounts of normal or abnormal repressor protein in a patient sample. For example, detection may utilize staining of cells or histological sections with labeled antibodies, performed in accordance with conventional methods. Cells are permeabilized to stain intracellular molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a flourescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

Alternatively, one may focus on the expression of the repressor protein. Biochemical studies may be performed to determine whether a sequence polymorphism in an coding region or control regions is associated with disease. Disease associated polymorphisms may include deletion or truncation of the gene, mutations that alter expression level, that affect the activity of the protein, etc.

Changes in the promoter or enhancer sequence that may affect expression levels of repressor protein can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc. that provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express repressor protein may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985), Science 239:487, and a review of techniques may be found in Sambrook, et al. Molecular Cloning: A Laboratory Manual, CSH Press 1989, pp.14.2-14.33. Alternatively, various methods are known in the art that utilize oligonucleotide ligation as a means of detecting polymorphisms, for examples see Riley et al. (1990), Nucl. Acids Res. 18:2887-2890; and Delahunty et al. (1996), Am. J. Hum. Genet 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods, and the sequence of bases compared to a wild-type sequence. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Single strand conformational polymorphism (SSCP) analysis, denaturing gradient gel electrophoresis (DGGE) and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations may be based on the functional or antigenic characteristics of the protein. Protein truncation assays are useful in detecting deletions that may affect the biological activity of the protein. Various immunoassays designed to detect polymorphisms in proteins may be used in screening. Where many diverse genetic mutations lead to a particular disease phenotype, functional protein assays have proven to be effective screening tools. The activity, e.g. TERT repressor functionality, of the encoded protein may be determined by comparison with the wild-type protein.

Diagnostic methods of the subject invention in which the level of expression is of interest will typically involve comparison of the nucleic acid abundance of a sample of interest with that of a control value to determine any relative differences, where the difference may be measured qualitatively and/or quantitatively, which differences are then related to the presence or absence of an abnormal expression pattern. A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

The following examples are offered by way of illustration and not by way of limitation.

### Experimental

I. Deletion Experiments

118 deletions of the minimal telomerase promoter (as defined by Takahura et al., Cancer Res. (1999) 59:551-7) were constructed to find regions within the telomerase promoter that contain potential repressor sites. These deletions ranged in size from 10 to 300 bases. Each deletion version of the minimal promoter was tested for its ability to express SEAP in MRC5 and HELA cells. Several of the deletions, all mapping about 50-100 bases upstream of the telomerase translation initiation codon (ATG), showed ˜10 fold increased expression. The highest expression in MRC5 was obtained with the deletion called 11K. This 30 base deletion includes bases −48 to −77 relative to the translation initiation codon ATG. However, a similar deletion, called 12K, that includes bases −48 to −57 results in 500 fold less expression. On the other hand, when 11K and 12K were compared in HELA, they both gave equivalent amounts of expression. The repressor site in this region of the TERT minimal promoter therefore is contained, or overlaps with, the 20 bases present in 12K and absent in 11 K (i.e. −58 to −77).

To identify more specifically the bases that make up this repressor site, additional deletions were made. Each deletion is 10 bases long with 7 to 8 base overlaps between consecutive deletions. The deletions were made in the minimal telomerase promoter in pSS120. Each deletion mutant was independently made three times and all deletions were transiently transfected into MRC5 (telomerase negative normal cells) and HELA (telomerase positive immortal cells).

A portion of the 5′ untranslated region is shown below, from −77 to 1, the start of translation (SEQ ID No:2). The Site C repressor site extends from −69 to −58, as shown.

| | |
|---|---|
| CTCCTCGC GGCGCGAGTT TCAGGCAGCG | SEQ ID NO: 2 |
| CTGCGTCCTG CTGCGCACGT GGGAAGCCCT | |
| r\overline{epressor site} (−69 to −58) | |
| GGCCCCGGCC ACCCCCGCGA | |
| \| | |
| start codon (1) | |

The repressor site is provided separately below as SEQ ID NO. 1.

| | |
|---|---|
| SEQ ID NO: 1 | GGCGCGAGTTTC |

The expression levels were measured using the Secreted Alkaline Phosphatase Assay (SEAP) system commercially available from Clontech Laboratories, Inc. (Palo Alto, Calif.). The results are shown below.

| Deletion | MRC5 | HELA |
|---|---|---|
| NONE (control) | 0.1931 | 78.3076 |
| −104 to −95 | 0.19 | 78.30 |
| −102 to −93 | 4.92 | 73.97 |
| −99 to −90 | 1.19 | 86.95 |
| −97 to −88 | 1.69 | 97.94 |
| −94 to −85 | 8.06 | 89.6 |
| −92 to −83 | 7.89 | 89.86 |
| −89 to −80 | 12.00 | 93.91 |
| −87 to −78 | 7.26 | 59.74 |
| −84 to −75 | 7.77 | 85.48 |
| −82 to −73 | 4.83 | 99.4 |
| −79 to −70 | 3.79 | 73.34 |
| −77 to −68 | 17.15 | 82.26 |
| −74 to −65 | 34.44 | 78.99 |
| −72 to −63 | 33.22 | 123.8 |
| −69 to −60 | 33.15 | 133.56 |
| −67 to −58 | 56.98 | 97.74 |
| −64 to −55 | 21.82 | 127.32 |
| −62 to −53 | 4.60 | 108 |
| −59 to −50 | 19.58 | 103.1 |

The column of deletions indicates the bases that were deleted in the repressor site, which is indicated relative to the AUG start codon. The columns for MRC5 and HELA show the level of expression observed for each deletion, reported as a percentage of the SV40 early promoter, which was used to normalize the two cell lines.

The data demonstrate that the deletion from “−67 to −58” gave a reading of 56.9852, as compared to a reading of 0.193109 in the control cells with no deletion in the promoter, giving an increase of 295 fold higher expression. This same deletion gave only 97.746 in HELA cells, compared to the undeleted control value of 78.3076, resulting in a 1.25 fold higher expression. This indicates that a repressor function operates in MRC5 cells to repress expression of the wild type telomerase promoter. When the expression level of deletion “−67 to −58” in MRC5 is compared to the wild type promoter in HELA it is observed that the deletion resulted in almost as much expression as the levels observed in HELA that are sufficient to maintain telomere length. That is, the expression of the deletion in MRC5 was 59.9852/78.3076=77% of the wild type in HELA. This indicates that depressing the repressor in MRC5 allows for sufficient amounts of telomerase expression to maintain the length of the telomeres in the cells during cell division, and to stop cellular aging in the cells.

II. Identification of Site C Binding Protein in U937 Cells

U937 cells are telomerase positive. However, treatment with the drug TPA causes them to become telomerase negative. Gel shift assays were performed using the Site C oligo and nuclear extracts from either non-treated or TPA treated U937 cells. The extract from the TPA treated U937 cells was observed to contain much more of the protein that binds Site C than the extract from the untreated cells. This finding is consistent with the mechanism that TPA treated U937 cells express more of the repressor protein that turns off the telomerase promoter and, resulting in these cells being telomerase negative.

III. One-Hybrid Screening Assay

The following experiments utilized the one-hybrid-assay system (purchased from Clontech Laboratories, Inc., Palo Alto, Calif.) to identify repressor factors that bind to the Site C sequence of the TERT promoter.

For the one hybrid assays utilized in the present invention, constructs were prepared with multiple tandem copies of the Site C bait element and then inserted upstream of a reporter gene in an appropriate vector (GAL4 AD vector). The reporter construct was subsequently integrated into the yeast genome to create a new yeast reporter strain. The yeast strain was transformed with an activation domain (AD) fusion library to screen for proteins containing DNA-binding domains (DNA-BD) that interact with the bait DNA sequence. Binding of an AD/DNA-BD hybrid protein to the bait sequence results in activation of the reporter gene transcription.

To screen a library for a cDNA encoding a binding protein that interacts with the bait sequence (Site C binding site), the modified yeast reporter strain was transformed with an activation domain (AD) fusion library, and then the transformants were plated on selective medium. If an AD fusion protein interacted with the bait DNA sequence, the HIS3 reporter gene was expressed, allowing colony growth on minimal medium lacking histidine. A HIS3, lacZ double reporter strain was used, allowing a β-galactosidase assay to be performed to confirm AD protein interaction with the bait element to eliminate false positives. Compatible cDNA libraries for the one hybrid assay system were purchased from Clontech Laboratories Inc. (Palo Alto, Calif.).

The Site C bait was designed to minimize the possibility of known yeast transcription factors binding to the bait element. For this reason, only a partial sequence of Site C was used as the bait element. Five tandem repeats of Site C were used to further increase the mimicking of the positive p53 bait controls.

The following partial sequence of Site C was used as bait, GGCGCGAGTTT (SEQ ID NO:3). The bases AG were inserted between each site C repeat. These changes in the bait sequence were made to minimize the similarity of this sequence to the consensus sequences of known yeast transcription factors. This modified bait sequence was tested in a transient transfection expression assay using human lung fibroblast cells (MRC5) to show that the Site C repressor factor(s) still bound to this shortened Site C. This shortened Site C was observed to cause significant repression in MRC5, but not as much as the complete Site C (SEQ ID NO: 1).

The Bait was made as a double strand oligonucleotide and was cloned into the EcoR1-XHO1 sites of pHisi, pHisi-1, and p53Blue. FIG. 1 shows a Site C bait fragment used in the one hybrid assays which contains five tandem repeats of an 11 base telomerase promoter fragment that includes 11 out of 12 bases of Site C.

Three different libraries were screened using the Site C bait sequence. The libraries were, human lung, human liver and human placenta (all purchased from Clontech Laboratories Inc., Palo Alto, Calif.). Each library was transformed into yeast reporter strains (containing the Site C bait sequences) and plated onto SD/-His/-Leu agar plates supplemented with 30 mM 3-amino-1,2,4-triazole media, as described by the vendor. The libraries were each plated at a density of ˜60,000 colonies per 150×15 mm plate. The lung library was plated on 50 plates (3×10⁶ total colonies) and the liver and placenta libraries were each plated on 300 plates (18×10⁷ total colonies each).

Approximately 150 large colonies were observed to grow on the plates after 5-6 days incubation at 30° C. Thirty of these colonies were observed on the plates containing the lung library while the plates containing the liver and placenta libraries each produced 60 large colonies. All large colonies were picked and tested for β-galactosidase activity. Three colonies from the lung library,13 colonies from the placenta library, and 25 colonies from the liver library tested positive for β-galactosidase activity.

These 41 colonies were grown in culture and plasmid DNA was isolated from each one. The plasmid DNAs were then transformed into E. coli DH5alpha, and the bacterial colonies were miniscreened with EcoR1 and XHO1. The miniscreens showed that the plasmids were not random isolates. Several of the plasmids had similar DNA restriction fragment patterns and were grouped accordingly into families. Five families of plasmids were observed. One family contained five EcoR1-XHO1 restriction fragments (8000 bp, 1100 bp, 500 bp, 300 bp, 150 bp). This family was found in all three libraries. Two families contained one fragment of approximately 1700 bp and 800 bp each and the remaining two families each had a unique sequence. The presence of “families” shows that the one-hybrid screen selected for specific cDNAs in the libraries.

IV. Backscreening of Positive Clones

2-4 plasmids from each family, plus a control plasmid expressing p53, were transformed into the following yeast strains; the original host strain that was used to screen the libraries and identify these plasmids, a control host strain containing a bait sequence for p53 (control does not contain the Site C bait).

All of the plasmids re-tested positive in the original host strain and tested negative in the p53 control host strain.

V. Identification of Repressor Proteins

A. ZNF140

1. DNA Sequences

The DNA sequences identified from the following plasmids: CF36-2 (from liver), CF36-L40 (from lung), and CF36-P6 (from placenta); are presented as SEQ ID NO. 4, 5, and 6 respectively.

| | |
|---|---|
| AAAGACTTTTCACCAAAAAATGTCATTTATGATGACTCATCCCAGTATTTGATCATGGAAAGAATTCTAAGTCAAGGCCC | SEQ ID NO. 4 |
| TGTGTATTCCAGTTTTAAAGGAGGCTGGAAATGCAAGGATCATACTGAGATGCTGCAAGAAAATCAGGGATGTATTAGGA | |
| AAGTAACAGTCTYTCATCAAGAARCCCTGGCTCAACATATGAATATCAGTACTGGGGAGAGGCCCTATGGATGCCATGAA | |
| TGTGGAAAAACTTTTGGTCGACGCTTTTCCCTGGKGTTACACCAGAGGACTCATACTGGAGAGAAACCATATGCATGTAA | |
| GGAATG...................AGTGATTTGGGAATGCCTTCATTTACAAQGCAATACTTAAATTTTAAQACTCTTG | |
| TAGGARAAAAAGCJACTGMATAAATGAATGTAGAGTGACYTTTTGCAATATTTCJAACCTATATCAJAGAATTACJCTGT | |
| GGGRAAACTACCJTTGTAATAAGMGTAGCAAAATCTCCTTARAQATYTGAAAAGTCATACTGGATGGAATCTGTAGGAAA | |
| CGGTTYTATTTTMARGGAAGGGGGATTCCYTTTTGTTTTTTAAGTGAATTCARAAAATGTTATAAATAAATCTTTTGGTT | |
| TQTTATAAAAAAAAAAAAAAAA | |
| TGGAAATGCAAGGATCATACTGAGATGCTGCAAGAAAATCAGGGATGTATTAGGAAAGTAACAGTCTCTCATCAAGAAGC | SEQ ID NO. 5 |
| CCTGGCTCAACATATGAATATCAGTACTGTGGAGAGGCCCTATGGATGCCATGAATGTGGAAAAACTTTTGGTCGACGCT | |
| TTTCCCTGGTGTTACACCAGAGGACTCATACTGGAGAGAAACCATATGCATGTAAGGAATGTGGCAAAACCTTTAGCCAG | |
| ATTTCAAACCTTGTGAAACACCAAATGATACATACTGGAAAGAAACCCCATGAGTGTAAGGACTGTAATAAAACATTCAG | |
| TTACCTTTCATTTCTTATTGAACACCAGAGAACGCACACTGGGGAGAAACCTTATGAATGTACTGAGTGTGGAAAGGCCT | |
| TTAGCCGTGCCTCCAACCTCACTCGACATCAGAGAATTCACATAGGAAAGAAACAATATATATGTAGGAAATGTGGTAAA | |
| GCATTTAGCAGTGGCTCAAAACTCATTCG | |
| .........CCAGGAAAGAATJCATATCCAATAGATTGGAGAAAGCCAGAGATTAGCCCTCATTCCGCATCTGTCAACCA | |
| GGACAGAATGCATGGCAAGGGATGAGCTTTACAAAGATGATGCJCTTTGGAGATCAGAAAATTCATATTTAAGCAAAGTG | |
| ATACAAACJCAGTGATTTGGGAATGCCTTCATTTACAATGCAATACTTAAATTTTAATACTCTTGTAGGAGAAAAAGCJA | |
| CTGTATAAATGAATGTAGAGTGACTTTCTGCAATATTTCAAACCTATATCAGAGAATTACACTGTGGGAAAACTACCATT | |
| GTAATAAGTGTAGCAAAATCTCCTTAGATATCTGAAAAGTCATACTGGATGGAATCTGTAGGAAACGGTTCTATTTTGAG | |
| GGAAGGGGGATTCCTTTTTGTTTTTTAAGTGAATTCAGAAAATGTTATAAATAAATCTTTTGGTTTATTATAAAAAAAAA | |
| AAAAAAAA | |
| GAGAAACCATATGCATGTAAGGAATGTGGCAAAACCTTTAGCCAGATTTCAAACCTTGTGAAACACCAAATGATACATAC | SEQ ID NO. 6 |
| TGGAAAGAAACCCCATGAGTGTAAGGACTGTAATAAAACATTCAGTTACCTTTCATTTCTTATTGAACACCAGAGAACGC | |
| ACACTGGGGAGAAACCTTATGAATGTACTGAGTGTGGAAAGGCCTTTAGCCGTGCCTCCAACCTCACTCGACATCAGAGA | |
| ATTCACATAGGAAAGAAACAATATATATGTAGGAAATGTGGTAAAGCATTTAGCAGTGGCTCARAACTCATTCGCCACCA | |
| GATTACACATACTGGAGAGAAACCTTATGAATGCATTGAATGKGGGAAGGCATTTCGCCGTTTCTCACACCTTACTCGAC | |
| ATCAGAGCATYCATACAACCAAAACCCCGTATGAATGTAATGAATGKAGGAAAGCTTTCCGTGTCACTCATTTCTTATTA | |
| AACATCAAAAAATTCATGCTGGAGAAAAGCTCTATGAATG........AAAAAAAAAAAAAAAAAATGGAAATGCAAGGA | |
| TCATACTGAGATGCTGCAAGAAAATCAGGGATGTATTAGGAAAGTAACAGTCTCTCATCAAGAAGCCCTGGCTCAACATA | |
| TGAATATCAGTACTGTGGAGAGGCCCTATGGATGCCATGAATGTGGAAAAACTTTTGGTCGACGCTTTTCCCTGGTGTTA | |
| CACCAGAGGACTCATACTGGAGAGAAACCATATGCATGTAAGGAATGTGGCAAAACCTTTAGCCAGATTTCAAACCTTGT | |
| GAAACACCAAATGATACATACTGGAAAGAAACCCCATGAGTGTAAGGACTGTAATAAAACATTCAGTTACCTTTCATTTC | |
| TTATTGAACACCAGAGAACGCACACTGGGGAGAAACCTTATGAATGTACTGAGTGTGGAAAGGCCTTTAGCCGTGCCTCC | |
| AACCTCACTCGACATCAGAGAATTCACATAGGAAAGAAACAATATATATGTAGGAAATGTGGTAAAGCATTTAGCAGTGG | |
| CTCAAAACTCATTCG | |

2. Consensus Sequence Homology

SEQ ID NOS. 4, 5, and 6 contain overlapping sequences with one another, providing further evidence that these plasmids are from the same family and encode for a similar transcription factor which binds to site C of the minimal hTERT promoter. A search of the Genbank database identified SEQ ID NO.4, 5 and 6 as matching the Zinc Finger Protein 140 (ZNF140) DNA encoding sequence (genbank reference NM_{—}003440) or homologs thereof.

Two copies of the ZNF140 gene have been identified in the Human Genome. One copy resides on chromosome 12 at 12q24.32-q24.33, and the other is located on chromosome 5 at an unknown location. The TERT gene also maps to chromosome 5.

None of the clones which have homology to ZNF140 contain the entire coding sequence of ZNF140. All three of the clones in the ZNF140 family have at least 9 of the 10 zinc fingers, but none of them contain the KRAB repression domains. The sequence encoding the Zinc Finger Protein 140 (ZNF140) was detected in all three of the random cDNA libraries used for screening (human liver, lung and placenta). This data supports the present invention that ZNF140 or a similar protein is a transcription factor which represses telomerase expression by binding to site C.

The TERT repressor proteins of interest have an encoding nucleic acid sequence that is substantially the same as, or identical to, that sequence which encodes for ZNF140. A given sequence is considered to be substantially similar to the ZNF140 sequence if it shares high sequence similarity with the above described specific sequence, e.g. at least 75% sequence identity, usually at least 90%, more usually at least 95% sequence identify with the above specific sequence.

B. HKR3 Repressor Protein

1.

The DNA sequences identified from from the following plasmids: CF36-10 (from liver), CF36-P46 (from placenta), CF36-P52 (from placenta), CF36-P56 (from placenta), and P18 (from placenta); are presented as SEQ ID NOS. 7, 8, 9, 10 and 11 respectively.

| | |
|---|---|
| CACGTATGTGAGTTCTGCAGCCACGCCTTCACCCAAAAGGCCAATCTCAACATGCACCTGCGCACACACACGGG | SEQ ID NO. 7: |
| TGAGAAGCCCTTCCAGTGCCACCTCTGTGGCAAGACCTTCCGAACCCAAGCCAGCCTGGACAAGCACAACCGCA | |
| CCCACACCGGGGAAAGGCCCTTCAGTTGCGAGTTCTGTGAACAGCGCTTCACTGAGAAGGGGCCCCTCCTGAGG | |
| CACGTGGCCAGCCGCCATCAGGAGGGCCGGCCCCACTTCTGCCAGATATGCGGCAAGACCTTCAAAGCCGTGGA | |
| GCAACTGCGTGTGCACGTCARACGGCACAAGGGGGTGAGGAAGTTTGAGTGCACCGAGTGTGGCTACAAGTTTA | |
| CCCGACAGGCCCACCTGCGGAGGCACATGGAGAT.....................ATGGTGGTGGTGGCGCTGC | |
| ARCCGCCTGCARARCTGGAGGTGGGCTCGGCGGAGGTCATTGTGGAGTCCCTGGCCCAGGGCGGCCTGGCCTCC | |
| CAGCTCCCCGGCCARARACTGTGTGCAAAGGARAGCTTYQCCGGCCCAGGTGTCCTGGAGCCCTCCCTCATCAT | |
| CACAGCTGCTGTCCCCGAGGACTGTGACACATAGCCCATTYTGGCCJCCARAGCCCJCTTGGCCCCJCCCCTCA | |
| ATAAACCGTGTGGCTTTGGAAAAAAAAAAAAAAAAA | |
| CTCTGTGGCAAGACCTTCCGAACCCAAGCCAGCCTGGACAAGCACAACCGCACCCACACCGGGGAAAGGCCCTT | SEQ ID NO. 8: |
| CAGTTGCGAGTTCTGTGAACAGCGCTTCACTGAGAAGGGGCCCCTCCTGAGGCACGTGGCCAGCCGCCATCAGG | |
| AGGGCCGGCCCCACTTCTGCCAGATATGCGGCAAGACCTTCAAAGCCGTGGAGCAACTGCGTGTGCACGTCAGA | |
| CGGCACAAGGGGGTGAGGAAGTTTGAGTGCACCGAGTGTGGCTACAAGTTTACCCGACAGGCCCACCTGCGGAG | |
| GCACATGGAGATCCACGACCGGGTAGAGAACTACAACCCGCGGCAGCGCAAGCTCCGCAACCTGATCATCGAGG | |
| ACRARAAGATGGTGGTGGTGGCGCTGCAGCCGCCTGCARAGCTGGAGGTGGGCTCGGCGGAGGTCATTGTGGAG | |
| TCCCTGCCCAGGGCGGCCTGCCTCCAGCTCCCCGGCCAGARACTGTGTGCAAAGGAAAACTTC........... | |
| AAAAAAAAAAAAAAAAAAA | |
| GGGCACCGGGCCTCGAGCCGGAATGGCCTGCAGATGCACATCAAGGCCAAGCACAGGAATGAGAGGCCACACGT | SEQ ID NO. 9: |
| ATGTGAGTTCTGCAGCCACGCCTTCACCCAAAAGGCCAATCTCAACATGCACCTGCGCACACACACGGGTGAGA | |
| AGCCCTTCCAGTGCCACCTCTGTGGCAAGACCTTCCGAACCCAAGCCAGCCTGGACAAGCACAACCGCACCCAC | |
| ACCGGGGAAAGGCCCTTCAGTTGCGAGTTCTGTGAACAGCGCTTCACTGAGAAGGGGCCCCTCCTGAGGCACGT | |
| GGCCAGCCGCCATCAGGAGGGCCGGCCCCACTTCTGCCAGATATGCGGCAAGACCTTCAAAGCCGTGGAGCAAC | |
| TGSGTGTGCACGTCAAAACGGCACAAGGGGGTGAGGAAAGTTGAGTGCACCGAGTGTGGCTACAAATTTACCCG | |
| ACAGGCCCACTTGGGGAGGCACATGGAAATCCCGACCGGGTAAAAAACTACAACCCCG.........AAAAAAA | |
| AAAAAAAAAAAAA | |
| ATCAAACTTCATGGAGCCCCCAAGCCCCATGCATGCCCCACCTGTGCCAAGTGCTTCTGTCTCGGACAGAGCTG | SEQ ID NO. 10 |
| CAGCTGCATGAAGCTTTCAAGCACCGTGGTGAGAAGCTGTTTGTGTGTGAGGAGTGTGGGCACCGGGCCTCGAG | |
| CCGGAATGGCCTGCAGATGCACATCAAGGCCAAGCACAGGAATGAGAGGCCACACGTATGTGAGTTCTGCAGCC | |
| ACGCCTTCACCCAAAAGGCCAATCTCAACATGCACCTGCGCACACACACGGGTGAGAAGCCCTTCCAGTGCCAC | |
| CTYTGTGGCAAGACCTTYCGAACCCAAGCCAGCCTGGACAAGCACAACCGCACCCACACCGGGGAAAGGCCCTT | |
| CAGTTGCGAGTTCTGTGAACAGCGCTTMACTGAGAAGGGGCCCCTYCTGAGGCACGTGGCCAGCCGCCATYAGG | |
| AGGGCCGGCCCCACTTYTGCCAGATATGCGGCAAGACCTTYAAAGCCGTGGAGCAACTGGGTGTGCACGT.... | |
| .......AAAAAAAAAAAAAAAAAAAA | |
| GAAGCCCGGAATTGCATGAACCGCTCGGAACAGGTCTTCACGTGCTCTGTGTGCCAGGAGACATTCCGCCGAAG | SEQ ID NO:11 |
| GATGGAGCTGCGGGTGCACATGGTGTCTCACACAGGGGAGATGCCCTACAAGTGTTCCTCCTGCTCCCAGCAGT | |
| TCATGCAGAAGAAGGACTTGCAGAGCCACATGATCAAACTTCATGGAGCCCCCAAGCCCCATGCATGCCCCACC | |
| TGTGCCAAGTGCTTCCTGTCTCGGACAGAGCTGCAGCTGCATGAAGCTTTCAAGCACCGTGGTGAGAAGCTGTT | |
| TGTGTGTGAGGAGTGTGGGCACCGGGCCTCGAGCCGGAATGGCCTGCAGATGCACATCAAGGCCAAGCACAGGA | |
| ATGAGAGGCCACACGTATGTGAGTTCTGCAGCCACGCCTTCACCCAAAAGGCCAATCTCAACATGCACCTGCGC | |
| ACACACACGGGTGAGAATCCCTTCCAGTGCCACCTTTGG | |

2. Consensus Sequence Homology

SEQ ID NOS. 7, 8, 9, 10 and 11 contain overlapping sequences with one another, providing further evidence that these plasmids are from the same family and encode for a similar transcription factor which binds to site C of the minimal hTERT promoter. A search of the Genbank database identified SEQ ID NOS. 7, 8, 9, 10 and 11 as matching the zinc finger protein Human Kruppel-Related 3 (KR3) DNA encoding sequence (Genbank references U45325 and NM_{—}005341) or homologs thereof. The four clones encode a partial sequence of HKR3 located near the C-terminal end of the protein.

HKR3 has 9 to 11 zinc fingers and is a known repressor of transcription. HKR3 has been shown to bind the S and/or J elements of the MHC Class II DPA gene promoter (Mol Cell Biol., Vol. 14:8438-8450,1994). The sequence of the MHC II PDA S element is GATCCGCACCTTG (SEQ ID NO:12) and the sequence of the J element is GATCCCTTTACCCAGG (SEQ ID NO:13). Both of these MHC Class II DPA sequences show some homology to the Site C binding site of the TERT promoter.

One copy of the HKR3 gene has been identified in the Human Genome. HKR3 maps to chromosome 1 at 1p36.3 which is within a region of the genome commonly rearranged or deleted in human cancers.

This data supports the present invention that HKR3 or a similar protein is a transcription factor that represses telomerase expression by binding to site C. The TERT repressor proteins of interest have an encoding nucleic acid sequence that is substantially the same as, or identical to, that sequence which encodes for HKR3. A given sequence is considered to be substantially similar to the HKR3 sequence if it shares high sequence similarity with the above described specific sequence, e.g. at least 75% sequence identity, usually at least 90%, more usually at least 95% sequence identify with the above specific sequence.

C. ZFP161

1.

The DNA sequence identified from plasmids CF36-14, and CF36-15 (both from liver) are presented as SEQ ID NOS. 14 and 15 respectively. Another DNA sequence identified from plasmid P13 (from placenta) is presented as SEQ ID NO. 16.

| | |
|---|---|
| CAGACCCCTCAAGCCTTAACATTTAATGATGGGATGAGTGAAGTGAAAGATGAACAGACACCAGGCTGGACAAC | SEQ ID NO. 14: |
| AGCCGCCAGTGACATGAAGTTTGAGTATTTGCTTTATGGTCACCATCGGGAGCAGATTGCCTGCCAGGCGTGTG | |
| GGAAGACGTTTTCTGATGAAGGCAGATTGAGGAAGCATGAGAAACTCCACACGGCGGACAGGCCATTTGTTTGT | |
| GAAATGTGCACAAAAGGTTTCACCACACAGGCCCACCTGAAAGAACACCTAAAAATCCACACAGGATATAAGCC | |
| CTATAGCTGTGAGGTGTGTGGAAAATCATTTATCCGTGCCCCAGACTTAAAGAAGCATGAGAGAGTTCACAGTA | |
| ATGAAAGACCGTTTGCGTGCCACATGTGTGACAAAGCCTTCAAACACAAGTCTCACCTYAAGGATCATGAAAGA | |
| AGACACAGAGGGGAAAAGCCTTTTGTGTGTGGYTCCTGCACCAAGGCATTTGCCA................... | |
| ..TTTTQAGTGTGAAAAAAAAAAAAAAAAAAAAAAA | |
| CAGACCCCTCAAGCCTTAACATTTAATGATGGGATGAGTGAAGTGAAAGATGAACAGACACCAGGCTGGACAAC | SEQ ID NO. 15: |
| AGCCGCCAGTGACATGAAGTTTGAGTATTTGCTTTATGGTCACCATCGGGAGCAGATTGCCTGCCAGGCGTGTG | |
| GGAAGACGTTTTCTGATGAAGGCAGATTGAGGAAGCATGAGAAACTCCACACGGCGGACAGGCCATTTGTTTGT | |
| GAAATGTGCACAAAAGGTTTCACCACACAGGCCCACCTGAAAGAACACCTAAAAATCCACACAGGATATAAGCC | |
| CTATAGCTGKGAGGKGTGTGGAAAATCATTTATCCGTGCCCCAGACTTAAAGAAGCATGAGAGAGTTCACAGTA | |
| ATGAAAGACCGTTTGCGTGCCACATGTGTGACAAAGCCTTCAAACACAAGTCTYACCTYAAGGRTCATGAAAGA | |
| AGA....................CCTACCCAAAAGCTGTAGTCACACATCCTAAAGGCCAARCAAACCCJCCGG | |
| GATGGTGGGGGGTCTTGGARCCAAGCTYTTAGGTTCCTCTTATTTGGGGCAGMACCAGTCCATACCAGCTGCGA | |
| TTTGTGAGTGGACCTGTGGTAAGAAGAATAGAAAAGGCTCTCARARATAAGGTTTTTTACATGTGTAACAATCC | |
| CAAGATTTCCTAGATTAAAATYTTAATTGATTTTGAAATTGGATTTTTATTTAGAATCAAAATTAGGRCAAGAA | |
| CAGATAACTTCTTCARATACATTTGTGTAACTTTACAGAATGTCATCAAGCTTTGGGGCTCTGTGGGGCACATG | |
| ATTTATCCATAAAGGAGATGCAGTATGCTTACTTAAATTAATAAATTTAAAATCTTTTAAGTGTGAAAAAAAAA | |
| AAAAAAAAAA | |
| AGTGACATGAAGTTTGAGTATTTGCTTTATGGTCACCATCGGGAGCAGATTGCCTGCCAGGCGTGTGGGAAGAC | SEQ ID NO:16 |
| GTTTTCTGATGAAGGCAGATTGAGGAAGCATGAGAAACTCCACACGGCGGACAGGCCATTTGTTTGTGAAATGT | |
| GCACAAAAGGTTTCACCACACAGGCCCACCTGAAAGAACACCTAAAAATCCACACAGGATATAAGCCCTATAGC | |
| TGTGAGGTGTGTGGAAAATCATTTATCCGTGCCCCAGACTTAAAGAAGCATGAGAGAGTTCACAGTAATGAAAG | |
| ACCGTTTGCGTGCCACATGTGTGACAAAGCCTTCAAACACAAGTCTCACCTCAAGGATCATGAAAGAAGACACA | |
| GAGGGGAAAAGCCTTTTGTGTGTGGCTCCTGCACCAAGGCATTTGCCAAGGCATCTGATCTGAAAAGGCACGAG | |
| AACAATATGCACAGTGAAAGGAAGCAGGTTACCCCCAGTGCCATCCAGAGCGAGACAGAACAGTTGCAGGCGGC | |
| AGCGATGGCTGCGGAAGCAGAACAGCAGCTGGAGACGATAGYCTGTAACTAGAGGCGGTGGGACAGGGACACTT | |
| TGCCTGGAAAG........ | |

2. Consensus Sequence Homology

A search of the genbank database identified SEQ ID NOS. 14 and 15 as matching the DNA encoding sequence for Zinc Finger Protein 161 (ZFP161) and a homolog, Zinc Finger Protein 5 (ZF5) (genbank reference nos. NM_{—}003409 and D89859). The sequence identified (SEQ ID NO.14 and 15) in plasmids 36-14 and 36-15 was detected in the human liver cDNA library and encodes a partial sequence of ZFP161 and ZF5: The ZFP161/ZF5 gene has been identified in the Human Genome as mapping to chromosome 18 at p11.21-pter.

ZFP161 and ZF5 have five zinc fingers and both proteins are known to be repressors. ZF5 has been shown to bind DNA sites comprising the sequence: GSGCGCGR, where S stands for G or C, R stands for A or G (Obata T, et al, Biochem Biophys Res Comm, Vol 255, 528-534,1999). This ZF5 binding site sequence is found in the bait used in the One Hybrid Screen and matches 7 bases of Site C and one base of the spacer sequence used between Site C repeats as shown below:

| | |
|---|---|
| ZF5 site | |
| GSGCGCGR | |
| ******** | |
| GAATTCGGCGCGAGTTTAGGGCGCGAGTTTAGGGCGCGAGTTTAGGGCGCGAGTTTAGGGCGCGAGTTTAG. | (SEQ ID NOS: 17 & 18) |
| *********** *********** *********** *********** *********** | |
| Site C Site C Site C Site C Site C | |

This data supports the present invention that ZFP161 or a similar protein is a transcription factor which represses telomerase expression by binding to site C.

The TERT repressor proteins of interest have an encoding nucleic acid sequence that is substantially the same as, or identical to, that sequence which encodes for ZFP161. A given sequence is considered to be substantially similar to the ZFP161 sequence if it shares high sequence similarity with the above described specific sequence, e.g. at least 75% sequence identity, usually at least 90%, more usually at least 95% sequence identify with the above specific sequence.

It is evident from the above results and discussion that the subject invention provides important new nucleic acid compositions that find use in a variety of applications, including the establishment of expression systems that exploit the regulatory mechanism of the TERT gene and the establishment of screening assays for agents that enhance TERT expression. In addition, the subject invention provides methods of enhancing TERT expression in a cellular or animal host, which methods find use in a variety of applications, including the production of scientific research reagents and therapeutic treatment applications. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for modulating a binding event between Site C and a repressor protein, said method comprising:
contacting said Site C and/or said repressor protein with a modulatory agent under conditions sufficient for binding of between said Site C and repressor protein to be modulated.

2. The method according to claim 1, wherein said method is a method of inhibiting binding between said Site C and repressor protein.

3. The method according to claim 1, wherein said method is a method of enhancing binding between said Site C and repressor protein.

4. The method according to claim 1, wherein said binding event is an in vitro binding event.

5. The method according to claim 1, wherein said binding event is an in vivo binding event.

6. The method according to claim 1, wherein said repressor protein is a protein having a sequence that is substantially the same as or identical to a protein selected from the group consisting of ZNF140, HKR3 and ZFP161.

7. A method of modulating expression of TERT from a TERT expression system that includes a Site C binding site, said method comprising:
modulating TERT expression by contacting said system with a modulatory agent under conditions sufficient for binding between said Site C and a Site C repressor protein to be modulated.

8. The method according to claim 7, wherein said method is a method of inhibiting binding between said Site C and repressor protein.

9. The method according to claim 7, wherein said method is a method of enhancing binding between said Site C and repressor protein.

10. The method according to claim 7, wherein said binding event is an in vitro binding event.

11. The method according to claim 7, wherein said binding event is an in vivo binding event.

12. The method according to claim 7, wherein said repressor protein is a protein having a sequence that is substantially the same as or identical to a protein selected from the group consisting of ZNF140, HKR3 and ZFP161.

13. A method of enhancing expression -of TERT from a TERT expression system that includes a Site C site, said method comprising:
inhibiting Site C repressor protein TERT transcription repression of said expression system.

14-24. (canceled)

25. A method for extending the lifespan of a mammal, said method comprising:
administering to said mammal an effective amount of an agent that inhibits Site C repression of TERT transcription by interfering with binding of a Site C repressor protein to Site C or inhibiting protein-protein interactions of a Site C repressor protein with cofactors that participate in TERT repression.

26-28. (canceled)

29. A method of decreasing expression of TERT from a TERT expression system that includes Site C, said method comprising:
enhancing a Site C repressor protein TERT transcription repression.

30-38. (canceled)

39. A method of determining whether an agent reduces a Site C repressor protein repression of TERT transcription, said method comprising:
(a) contacting said agent with an expression system comprising a Site C sequence, said Site C repressor protein and a coding sequence under conditions such that in the absence of said agent, transcription of said coding sequence is repressed;
(b) determining whether transcription of said coding sequence is repressed in the presence of said agent; and
(c) identifying said agent-as an agent inhibits repression of TERT transcription if transcription of said coding sequence is not repressed in the presence of said agent.

40-47. (canceled)
